(19) United States
(12) Patent Application Publication
(10) Pub. No.: US 2016/0157725 A1
(43) Pub. Date: Jun. 9, 2016

(54) DEVICE, SYSTEM AND METHODS FOR ASSESSING TISSUE STRUCTURES, PATHOLOGY, AND HEALING

(71) Applicant: Luis Daniel Munoz, Windsor Locks, CT (US)

(72) Inventor: Luis Daniel Munoz, Windsor Locks, CT (US)

(21) Appl. No.: 14/962,745

(22) Filed: Dec. 8, 2015

Related U.S. Application Data
(60) Provisional application No. 62/088,809, filed on Dec. 8, 2014.

(51) Int. Cl. A61B 5/00; A61B 5/05; H04N 5/225; H04N 7/18; H04N 5/33

(52) U.S. Cl. CPC A61B 5/0077; H04N 7/185; H04N 5/33; H04N 5/2252; H04N 5/2256; A61B 5/0075; A61B 5/0086; A61B 5/0507; A61B 5/7425; A61B 5/7475; A61B 5/6898; A61B 5/0013; A61B 2560/0425; A61B 2576/00; A61B 2019/521; A61B 2560/0214

(57) ABSTRACT

Disclosed herein are portable handheld devices, systems, and methods for the evaluation of tissue pathology and the evaluation and/or monitoring of tissue regeneration. The handheld devices and systems perform laser speckle and hyperspectral imaging to assess tissue pathology and tissue regeneration. The device and system of the disclosure may also perform 3D surface reconstruction.

### CROSS-REFERENCE TO RELATED APPLICATIONS

This application is a non-provisional application of U.S. Provisional Application No. 62/088,809, titled WOUND EVALUATION AND EARLY DISEASE DETECTION SYSTEM, filed 8 Dec. 2014, the entire contents of which are incorporated herein by reference.

### BACKGROUND

1. Field of the Discovery

The description provides a device and system for assessing tissue disease, tissue healing, and tissue regeneration, and methods of use thereof.

2. Background Information

Annually, over 5 million patients in the US suffer from some form of tissue pathology and/or tissue pathology complication. Due to the increased demand for healthcare and insufficient supply of clinical resources and clinician experts, routine care for tissue diseases and preventive screenings for tissue and skin malignancies have been displaced to emergency departments, urgent care clinics, specialty clinics, home care services, and third party health services providers. As a result of the current shift in healthcare demands and policies, routine evaluations, assessments, and monitoring of tissue pathologies (e.g., chronic wounds, tissue and skin malignancies, and post-surgical wounds) and the monitoring and analysis of tissue healing and tissue regeneration are more likely to be managed at the point-of-care by clinical support staff (e.g., nurses or physician assistants) and/or caregivers who may lack sufficient medical expertise in this area to effectively report, manage, and treat the condition.

A vital step in medical examinations is to conduct visual inspection. In the context of evaluating and assessing tissue pathologies, such an examination, requires a trained eye in order to effectively define the characteristics that relate to specific pathologies, anatomical landmarks, tissue types, characteristics of tissue repair, and/or signs of complications, all of which will influence the status, diagnosis, and interventions that relate to the specific tissue disease. Examples of tissue diseases include, diabetic ulcers, pressure ulcers, decubitus ulcers, vascular ulcers, traumatic wounds, non-healing wounds, surgical wounds, malignancy related wounds/ulcers, radiation-induced wounds, while also including skin malignancies that may affect multiple layers of tissue (e.g., skin, soft tissue, muscle, etc.). Furthermore, the increased prevalence of chronic tissue diseases and management of these diseases have created a growing demand for new modular and portable diagnostic technologies that are able to effectively clinically assess and monitor disease and tissue healing and tissue regeneration, and thereby promote advances in management and therapeutic protocols. As it relates to tissue regeneration, there is a growing field of regenerative medicine that seeks to advance methods to promote tissue healing and to develop/create/generate new tissues, which in part may follow some of the same physiological processes described above and herein.

Tissue healing (e.g., in wound healing) is a complex process in which epidermis (outer layer of skin) and dermis (dense inner layer of skin) repairs itself after the tissue is damaged. The epidermis and dermis provide a protective barrier against the external environment. When the skin is broken, the tissue is repaired through a cascade of phases: blood clotting (homeostasis), inflammation, growth of new tissue (proliferation), and remodeling of tissue (maturation). The clot provides a barrier in the blood vessels that slows or prevents further bleeding Inflammation clears dead and damaged cells, as well as pathogens (e.g., bacteria, viruses, fungi, parasites, etc.) and debris. The proliferation phase includes angiogenesis (i.e. new blood vessel form from vascular endothelial cells), collagen deposition, granulation tissue formation (i.e., provisional extracellular matrix is formed by the excretion of collagen and fibronectin from fibroblasts), epithelialization (i.e., epithelial cells proliferate and migrate atop the wound bed, thereby providing a cover for the new tissue), and wound contraction (i.e., myofibroblasts decrease the size of the wound through contraction and ultimately apoptosis upon completion of the contraction phase). The remodeling phase includes the realignment of collagen along tension lines, and cells no longer required undergo apoptosis.

Tissue healing and regeneration (e.g., in wound healing) is susceptible to interruption and failure, which can result in chronic wounds. Wounds are predisposed to several complications such as hemorrhage, non-healing, infection, gangrene, poor scarring, and malignancy. Detecting and reducing tissue pathologies, as well as monitoring tissue regeneration, are important to hospital systems, health insurers, health providers and patients, since health care costs and morbidity related to complicated tissue pathologies has risen dramatically. The drivers for the increase in incidence and prevalence of tissue pathologies are: an increase in surgical procedures (approximately 240 million per year), penetrating wounds (approximately 5 million per year), pressure ulcers (approximately 3 million per year), burns (approximately 450 thousand per year), and other chronic diseases resulting in tissue pathology (e.g., vascular insufficiency and diabetes related ulcers), and skin malignancies (e.g., approximately 8 million per year in US).

Commercially available imaging modalities that have resulted from novel advances in micro-semiconductor technologies allow for the development of a faster, more compact, and portable imaging system for the analysis, assessment, and monitoring of tissue disease and tissue healing and regeneration, which can be implemented in a mobile and portable form in hospitals, medical facilities, as well as remote locations with limited resources. One problem is that often it is necessary to assess the tissue composition and oxygenation over a large area and in real or near-real time. A secondary problem relates to the limitations of a single imaging modality and technologies. A tertiary problem relates to the reliability of image data and interpretations as they relate to clinical care.

To date, known imaging technologies include hyperspectral imaging (HSI), laser speckle imaging/laser speckle contrast imaging (LSI/LSCI), tomographic imaging (TI), and thermographic imaging (TGI). As it relates to medical imaging, known technologies leveraging one or two of the aforementioned imaging modalities have been restricted to bulkier designs due to the use of multiple optical elements, while also having higher energy demands due to the use of scanning-based imaging sensors, required cooling systems, massive data processing, and light source selections, drivers, and controllers.

As a single imaging modality, medical HSI consists of fast image processing steps that do not require prior knowledge of the tissue or its metabolic state, but that can also take additional information from tissue into account if so desired. Medical HSI also has the ability of assessing microvascular changes in skin due to inflammation, blood perfusion, and other cellular alterations. Medical HSI also allows the quantitative monitoring of tissue therapies as a means of optimizing treatment on an individual basis or for the exploratory screening and optimization of new drugs. This may be useful in the staging of disease or in the monitoring of the results of a particular therapeutic regimen. Medical HSI provides additional information to the doctor that is not currently available and can be used along with other clinical assessments to make this decision.

Currently, many optical and non-optical technologies cannot provide easily accessible and interpretable imaging data in real-time. Medical HSI is one modality that solves that problem by processing the hyperspectral cubes in near real-time and presenting a high-resolution, pseudo-color image where color varies with tissue type and oxygenation (a marker of viability). Medical HSI transcribes 3D spectral information into one image preserving biological complexity via millions of color shades. The particular color and distinct shape of features in the pseudo-color image allow discriminate between tissue types such as tumor, connective tissue, muscle, extravasated blood, and blood vessels. The human eye expansion capabilities relate to the interpretation of color spectrums. The most common used color space in computer vision technology is the RGB color space because it deals directly with red, green, and blue channels that are closely associated with the human visual system, but another employed color space is the HSI (hue, saturation, intensity) color space which is based on human color perception and can be described by a color cone. The RGB and HSI color spaces can be easily converted from one to the other. A pseudo-color image transformation refers to mapping a single-channel image to a three-channel image by assigning different colors to different features. The principal use of pseudo-color is to aid human visualization and interpretation of grayscale images, since the combinations of hue, saturation, and intensity can be discerned by humans much better than the shades of gray alone. The technique of intensity (density) slicing and color coding is a simple example of pseudo-color image processing. If an image is interpreted as a 3-D function, this method can be viewed as one of painting each elevation with a different color.

Medical HSI main purposes include 1) expand human eye capabilities beyond the ordinary; 2) expand the human brain capabilities by pre-analyzing the spectral characteristics of the observable subject; 3) perform these tasks with real or near-real time data acquisition. The aim of the algorithm is to facilitate the human to diagnose and assess the condition of the observable subject.

A problem with medical HSI technologies is related to the known use of line-scan hyperspectral image sensors, which require a 20-30 second optical scan and/or longer scan of a region of interest, and require additional time to process, interpret, and reconstruct the visual data for presentation. The prolonged scanning and processing time result in a delayed analysis and interpretation, which can disrupt work-flow and delay time to care in settings that require real-time presentation of data for clinical management and support.

Another problem is that the current systems that incorporate the use of medical HSI tend to be bulkier, due to the multitude of optical elements required for scanning-based HSI sensors. The current systems are large portable systems built for in hospital portability, but are still too large for mobile, portable, and handheld applications.

An additional problem is that due to the complexity of the biological system, medical personnel want to have as much information as possible about a given case in order to make the most-reliable diagnosis. The current systems are restricted to one or two of the available imaging modalities and lack the ability to interface with a combination of systems including, mobile devices and software, cloud-based systems, and hospital based systems, which results in slower processing times, less than reliable analysis of tissues, limitations in the synthesis of actionable clinical data, and an inability to adequately monitor tissue disease, healing and regeneration in both hospital and remote (non-hospital) settings.

As another single imaging modality based on the use of coherent light, image detector, and algorithms, Laser Speckle Contrast Imaging (LSI/LSCI) and variations thereof, can serve to further investigate microvasculature, tissue oxygenation, distinct blood particulates, and in the computational 3D reconstruction of structures related to the observed anatomy. LSCI is a minimally invasive method used to image blood flow by illuminating chromophores (e.g., hemoglobin) within the blood in vivo and tracking their relative velocities in the blood as they flow with high spatial and temporal resolution by utilizing the interference effects of a coherent light source. Non-moving scattering particles in the media produce a stable speckle pattern, whereas movement of scattering particles causes phase shifts in the scattered light, and temporal changes in the speckle pattern. In LSCI, time integrated speckle pattern can be used to estimate blood flow in a tissue. Combining the blood flow and oxygenation information can provide a better estimate of underlying soft tissue activity and serve as an indirect indicator of metabolic dynamics.

Furthermore, a real-time portable and handheld laser speckle imager in a clinical or remote setting could be used to create personalized treatment plans based on frequent analysis of microvascular regeneration, structural changes, disease progression, and therapeutic efficacy monitoring. Furthermore, the image data can be used to create a 3-D construct of the microvascular networks, which can be used to aid medical simulations, investigations, and interventions. Lasers have been used as effective and cost efficient light sources for optical imaging applications. Lasers, however, introduce coherence effect noise (random constructive/destructive interference) which superimpose a speckle pattern over the signal, and thereby prevent generation of low-noise, high-brightness illuminations, which are required for example for evaluating tissue oxygenation in neural imaging. Two types of lasers diodes are edge-emitting laser diodes and VCSELs (vertical-cavity surface emitting lasers). Most imaging technologies still use Edge-Emitting Laser Diodes within their design. Edge emitting laser diodes produce a coherent light beam that is elliptically shaped beams, leading to higher levels of interference patterns (increased speckle patterns) and requiring multiple and stronger corrective optical elements. The optical beam properties, required corrective elements, shape design, and relatively larger size of edge emitting laser diodes have presented challenges in developing compact, efficient, and portable handheld laser speckle imager.

In regards to techniques and technologies related to 3D surface imaging has recently been more widely incorporated into consumer electronics in order to sense depth, dimensions of physical structures, and virtualization of the outside world (e.g., virtual reality gaming technologies). 3D surface imaging is achieved through a light sensor(s), depth sensor(s), and image detector(s). The image data can be analyzed and used to reconstruct 3D surface images of the objects, which is more superficial than the reconstructions achieved by LSCI (e.g., micrometers to millimeters). The accessibility of depth sensing technologies, cameras, and software in consumer electronic devices, such as a select group of mobile and gaming devices, has presented an opportunity to leverage 3D surface imaging and technologies to visualize tissue disease and architecture in a more interactive way.

In regards to techniques and technologies related to thermographic imagers, thermal imaging has been used to evaluate soft tissue characteristics such as, inflammation, infection (non-specific), and blood flow.

A common, but widely accepted, problem with current imaging devices is their size and significant cost (hardware, software, and associated support and training costs). Also, due to bulky, fixed implementation, subjects must be brought to the device—it cannot be brought to them. This limits the applications of optical imaging technologies. Thus, there is a need for a lower cost, portable, optical imaging system that is operable to generate useful imaging series, and can be applied to a variety of subjects, including in health care and biomedical research settings. Yet another problem with imaging devices is the need to use different imaging modalities in order to enable capturing of different types of images and a series of images captured over time for making a required diagnosis. Performing multiple images in series using different imaging modalities can significantly increase the time and costs for performing image analysis for a given subject. For instance, current image sensors and camera assemblies have board level interfaces that make them slower to process image data, require more energy to power, and present challenges to integrate multiple peripheral sensors and cameras, some examples of lower level interfaces are USB 2.0, FireWire, and GigE.

Therefore, there exists a need for an efficient, multi-modal, portable device and/or system that can perform approximately real-time, automated imaging assessments of biological tissue to evaluate, monitor and detect tissue disease progression and/or tissue healing and regeneration, and to combine said findings with pertinent clinical, anatomical, and physiological data, to improve clinical analysis and assessments of tissue diseases made in hospital and remote settings with and/or without extensive training and experience.

### SUMMARY

The present description relates to a portable handheld device, a portable automated, modular system, and imaging and clinical methods for the early evaluation, disease detection, clinical assessment, and/or monitoring of tissue healing, regeneration and disease progression. Embodiments of the present disclosure will reduce the number of complications that result from tissue diseases and prolonged tissue healing by promoting earlier detection and interventions. Embodiments of the present disclose can be easily implemented in remote locations with few resources with little training and/or experience with evaluating tissue disease and tissue healing assessments such as, the management of chronic ulcers and non-healing wounds. As such, embodiments of the present disclosure help caregivers navigate through the management and treatment of tissue pathologies and monitoring of tissue healing progression and tissue disease progression.

According to an object of the present disclosure, a mobile or portable device for performing imaging of biological tissue is provided. The device can be handheld or configured to be attached to a stand, another imaging device, or any other relevant pertinent apparatus, device or system. The device can comprise a housing including: a source assembly, at least one acquisition device for capturing images of a region of interest of a subject, and a control module. The source assembly has at least one light source. The at least one acquisition device includes a hyperspectral image sensor, and optionally at least one of an IR image sensor and/or a camera, depth sensor and/or pertinent optics, RGB sensor and/or camera, and a thermographic sensor and/or a camera. The control module is configured to communicate with a mobile device with a camera and to process raw laser speckle image data from the camera or the handheld device, raw digital picture data from either the camera or the hyperspectral image sensor, and raw hyperspectral image sensor data to evaluate tissue pathology and to monitor tissue healing and regeneration of the region of interest. The device is a portable and attachable device, which when attached to a mobile device or independent of any mobile attachment, is a handheld multimodal imager.

In an embodiment, the at least one light source is at least one of a light emitting diode (LED) and a laser. The at least one light source may comprise two light sources that emit light at a wavelength in a range of about 400 nm to about 2,500 nm, and about 600 nm to about 2,500 nm. In a particular embodiment, the about 400 nm to about 2,500 nm wavelength light is emitted from a LED and the about 600 nm to about 2,500 nm wavelength light is emitted from a laser. The laser can be a laser diode or vertical cavity surface emitting laser (VCSEL).

In another embodiment, the portable handheld device further comprises compact optical elements, such as a micro-diffuser lens, located within a light path of at least one of the at least one laser. The handheld device may further comprise a polarizing film and/or lens located within a light path of at least one of the at least one LED. The portable handheld device may further comprise mechanical, electronic, and digital features and components to integrate and/or attach and/or interface with larger mechanical systems such as, medical robotics, to serve as a distinct and modular medical imaging and vision system.

In certain embodiments, the hyperspectral image sensor is a snapshot mosaic hyperspectral imaging sensor, which can capture a hyperspectral datacube without translational movement, unlike the aforementioned line-scan systems. New commercially available snapshot hyperspectral imaging technologies enable real-time, dynamic applications, and can use monolithically integrated Fabry-Perot (FP) filters on top of an image sensor for low cost, compactness and high speed. A snapshot hyperspectral imager may present a practical solution for fast, compact, user-friendly and low cost spectral imaging cameras and consists in monolithically integrating optical interference filters on top of CMOS-based image sensors to produce a spectral imager with high temporal resolution.

In an embodiment, the hyperspectral image sensor is assembled as a high speed camera that transfers data at up to 5 Gbit/s (625 MB/s) (e.g., using the SuperSpeed transfer mode/process utilized by the USB 3.0 standard. The hyperspectral image sensor can be a snapshot mosaic hyperspectral image sensor. In another embodiment, the hyperspectral image sensor transfers data at up to 10 Gbits/s (1.25 GB/s) (e.g., using the SuperSpeed+ transfer mode/process utilized by the USB 3.1 standard). The device can also include a data port or plug, e.g., a USB 3.0 or 3.1 compliant data port/plug. The data port or plug can be utilized for peripheral imagers.

The portable handheld device may further comprise at least one of a rechargeable battery, a camera of the device, a display of the device, and an input device.

In an embodiment, the control module includes a heterogeneous computational board with a field-programmable gate array (FPGA), a graphic processing unit (GPU), and a central processing unit (CPU). In other embodiments, the FPGA is configured with functional algorithms to extract, process, and/or analyze/interpret the hyperspectral image sensor data and/or the laser speckle image data. In another embodiment, the GPU is configured with functional algorithms to extract, process and/or analyze/interpret the hyperspectral image sensor data and/or the laser speckle image data. In another embodiment, the GPU and FPGA are configured with functional algorithms to co-process and/or analyze, and interpret at least one of the hyperspectral image data, the laser speckle image data, the 3D surface image data, and thermographic image data. In a further embodiment, the control module is configured to register a plurality of processed data and a plurality of raw data.

In a further embodiment, the control module is configured to overlay at least two of the plurality of processed data and the plurality of raw data, and to transmit the overlay to a screen of the mobile device for display or to a screen of the device for display or to a screen of a remote device located at a remote location for display.

The mobile handheld device may further comprise microcontrollers configured to control the plurality of acquisition devices and the source assembly during the imaging of the region of interest.

In a further embodiment, the acquisition devices, optical elements and light source assemblies, and imaging algorithms may be configured and microcontrolled as distinct modes for image acquisition.

According to another object of the present disclosure, a system for performing imaging of biological tissue is provided. The system comprising: a mobile device having a camera for capturing images of a region of interest of a subject including at least one of raw laser speckle images, digital images, and 3D images; and a housing. The housing may comprise: a source assembly, at least one acquisition device for capturing images of the region of interest, and a control module. The source assembly includes at least one light source. The at least one acquisition device includes at least one of: a snapshot hyperspectral image sensor, an IR sensor and/or a camera, a RGB sensor and/or a camera, and a thermographic sensor and/or a camera. The control module is configured to receive and process the raw laser speckle images, the raw hyperspectral image sensor data, depth sensing data for 3D surface images, and the raw thermographic sensor data if present. The control module is configured to communicate with the mobile device, and includes a processor comprising non-transitory computer readable medium configured to analyze the raw laser speckle images from the camera, and raw hyperspectral image sensor data (and the thermographic sensor data if present) to detect or assess tissue pathology.

In an embodiment, the at least one light source comprises a LED that emits light at a wavelength in a range of about 400 nm to about 2,500 nm, and a laser that emits light at a wavelength in a range of about 600 nm to about 2,500 nm. In an embodiment, the at least one light source is at least one of a LED and a VCSEL.

In a particular embodiment, the control module includes a heterogeneous computational board with a field-programmable gate array (FPGA), a graphic processing unit (GPU), and a central processing unit (CPU). In an embodiment, the FPGA is configured to extract/process and/or analyze the hyperspectral image sensor data and/or the laser speckle image data. In another embodiment, the GPU is configured to process and/or analyze the hyperspectral image sensor data and/or the laser speckle image data. In an embodiment, the FPGA and GPU are configured to co-process and/or analyze the hyperspectral image sensor data and/or the laser speckle image data with variable combinations of processing algorithms distributed between the FPGA and GPU.

In another embodiment, the control module is configured to register a plurality of processed data and a plurality of raw data. The control module may be configured to overlay at least two of the plurality of processed data and the plurality of raw data, and to transmit the overlay to a screen of the mobile device, a screen of the housing, or a screen of a remove device for display.

According to another object of the present disclosure, a method for evaluating tissue pathology and monitoring tissue regeneration is provided. The method comprising: providing a handheld device or the system with or without a mobile device with or without a software analytics program. For example, the method comprises providing a mobile device combined with a handheld imager and software analytics program, the handheld imager having: a camera for acquiring raw laser speckle images; a depth sensor and a camera for acquiring 3D surface images; a source assembly including at least one light source; at least one acquisition device including a hyperspectral image sensor, and a control module. The computer analytics program can perform the aggregation of image data, transfer of image data, analysis of image data, storage of image data, pattern recognition of image data, synthesis of image data reports for clinical care, patient and clinical meta-data, direct and/or indirect interface with healthcare related software programs and portals, clinical meta-data reports, statistical modeling, and machine learning capabilities. The method further comprises acquiring the raw laser speckle image and hyperspectral image and 3D surface image of a tissue region of interest of a subject in need thereof; and analyze and monitor the tissue region of interest, and optionally diagnosing, triaging, intervening, and/or treating the subject having tissue pathology.

In a particular embodiment, analyzing the tissue region of interest includes processing the raw laser speckle image and the raw hyperspectral image. In another embodiment, analyzing the tissue region of interest includes overlaying at least two of a plurality of processed data images and/or a plurality of raw data images; and transmitting the overlay to a screen of the mobile device for display and/or screen of a remote device located at a remote location for display.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages, objects and embodiments are expressly included within the scope of the present invention.

The publications and other materials used herein to illuminate the background of the invention, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention.

FIG. 1. An illustration of the handheld device in accordance with an embodiment of the present disclosure.

FIG. 2. An illustration of a handheld device in accordance with an embodiment of the present disclosure.

FIG. 3. An illustration of a handheld device in accordance with an embodiment of the present disclosure and a mobile device being inserted into the cradle of the device.

FIG. 4. An illustration of a handheld device with a mobile device located in a cradle of the handheld device with a mobile device or a system in accordance with an embodiment of the present disclosure.

FIG. 5. An illustration of a handheld device in accordance with an embodiment of the present disclosure and a mobile device being inserted into the cradle of the device.

FIG. 6. An illustration of a handheld device in accordance with an embodiment of the present disclosure wherein the retention pieces are attached by, e.g., screws.

FIGS. 7A, 7B, 7C, and 7D. Illustrations of several perspectives of a hyperspectral imager used in a device of the present invention.

FIGS. 8A, 8B, 8C, and 8D. Illustration of several perspectives of a light source used in a device of the present invention.

FIG. 9. An illustration of a heterogeneous computational board.

FIG. 10. An illustration of a system 200 in accordance with an embodiment of the invention.

FIG. 11. A flow diagram of the method in accordance with an embodiment of the invention.

FIG. 12. Diagram of the flow of data saving, retrieval, and analysis in accordance with an embodiment of the invention.

### DETAILED DESCRIPTION

The following is a detailed description of the disclosure provided to aid those skilled in the art in practicing the present disclosure. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure. All publications, patent applications, patents, figures and other references mentioned herein are expressly incorporated by reference in their entirety.

Presently described are devices, systems, and methods that relate to the surprising and unexpected discovery that one can utilize a combination of virtual and augmented imaging techniques, sensors, and data for real-time and non-invasive portable medical imaging, for example, spectral, backscatter, 3D images, photographic images, to evaluate tissue structures, pathology, and monitor tissue healing and regeneration. That is, embodiments of the present invention provide a three dimensional reconstruction of visual and anatomical data and augmentation of color spectral patterns that serve as spectral fingerprints of distinct tissue types and characteristics acquired from an area of interest, which facilitates an early evaluation of tissue pathology (e.g., malignancies, ulcers, eschars, scabs, wounds, etc.) and tissue healing and regeneration in, for example, wounds, thereby providing early disease detection, promoting earlier interventions and reducing the number of complications with tissue pathologies and the number of advanced interventions that result from wound complications and other tissue pathologies. Embodiments of the present disclosure provide a device and system for an early evaluation of tissue healing and regeneration and tissue pathology, as well as the detection of tissue diseases and complications, for example, as wounds progress through the healing process. Embodiments of the present disclosure can easily be implemented in remote locations, i.e., regions with little resources and/or medical expertise, with little training and/or experience with evaluating tissue pathology and tissue healing and regeneration (e.g., in wound healing). Furthermore, embodiments of the present disclosure can be utilized in any setting where tissue pathology and tissue healing regeneration is observed, managed and/or treated. For example, the device and system can be provided to caregivers, physician extenders, physicians, technical health providers, and other clinical support staff, thereby providing an early evaluation and disease detection tool that integrates and aggregates visual and clinical data patterns, medical evaluations, clinical meta-data, clinical logs, subjective scales, and sensor based analysis to help navigate treatment and management of tissue pathology detection, evaluation, and healing and regeneration. In particular, embodiments of the present disclosure can be utilized to assess decubitus ulcers, diabetic ulcers, surgical procedures, skin and soft tissue malignancies, burns, chronic wounds, non-healing wounds, normal tissue healing and tissue regeneration.

Where a range of values is provided, it is understood that each intervening value between the upper and lower limit of that range and any other stated or intervening value in that stated range is encompassed within the disclosure. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the 10 United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

The handheld device, system, and methods of the disclosure are effective at evaluating tissue structures, tissue pathology, and tissue regeneration, detecting the presence of disease, and monitoring the progress of a detected disease and/or tissue healing and regeneration progress. The embodiments of the disclosure require little training or experience in assessing tissue pathology and tissue healing and regeneration. The present disclosure provides for a device, system, and methods that may be implemented in hospital and/or remote locations with limited health resources and/or experts.

According to an aspect of the invention, a mobile device (e.g., a handheld device) for performing imaging of biological tissue is provided. The device can comprise a housing including: a source assembly, at least one acquisition device for capturing images of a region of interest of a subject, and a control module. The source assembly having at least one light source, and the at least one acquisition device includes a hyperspectral image sensor. In an embodiment, the control module is configured to communicate with a mobile device with a camera and to process raw laser speckle image data from the camera, raw digital picture data from either the camera or the hyperspectral image sensor, and raw hyperspectral image sensor data, and 3D surface image data to evaluate tissue pathology and tissue healing and regeneration of the region of interest. In an embodiment, the control module is configured to communicate with the at least one light source and the at least one acquisition device. For example, the control module is linked with the mobile device, the at least one light source, and/or the at least one acquisition device. The link can be a wireless link (as described in greater detail below) and/or direct link (e.g., a wire, a data port on the mobile device that directly interacts with a complimentary data plug on the device or system, or vice versa), or by any other means known in the art. Exemplary data ports and plugs/cables connections include universal serial bus (USB) (e.g., USB 1.x, USB 2.0, USB 3.0, USB 3.1, USB Type-C, micro USB, mini USB, firewire, eSATA, etc.).

In an embodiment, the device produces a combination of 2D surface, 3D surface and/or, 3D sub-surface image reconstruction of visual and anatomical data. That is, the device can produce a 3D stereoscopic digital representation of tissue structures and pathology, for example, wound borders, contour, and layers. Also, the device can produce a 3D stereoscopic digital representation of tissue structures, pathology and microvasculature, for example, capillaries and smaller veins and arteries that approximate the surface of the skin and/or tissues within micrometers to millimeters of depth. The device can also determine degrees of tissue oxygenation, spectral signatures, cellular and molecular signatures of a particular type of tissue and tissue pathology (e.g., a particular type of ulcer or malignancy), and/or provide a tissue spectral fingerprint that can be used in evaluating tissue pathology and/or tissue healing and regeneration. The device can also determine blood flow, detect visual signs of complications, and/or estimate healing time of tissue pathologies within the region of interest. In another embodiment, the device can provide a tissue viability analysis based on, for example, subcutaneous red blood cell concentration in the imaged region and/or by evaluating variations in light reflectance between healthy and diseased tissues through the use of polarized light. The device of the present disclosure can also provide the unique ability to add peripheral/additional sensors to compliment remote healthcare applications as required, based on the heterogeneous computational board level configurations and computer based analytics.

In some embodiments, the raw laser speckle image data, the raw hyperspectral image sensor data, and the raw digital picture data (e.g., an RGB picture) are acquired at substantially the same time. In other embodiments, the raw laser speckle image data, the raw hyperspectral image sensor data, the raw digital picture data (e.g., an RGB picture), and the 3D surface image data are acquired sequentially (e.g., the order could be one of the following: the raw laser speckle image data, the raw hyperspectral image sensor data, the raw digital picture data, and 3D surface image data; the raw laser speckle image data, 3D surface image data, the raw hyperspectral image sensor data, and the raw digital picture data; 3D surface image data, the raw laser speckle image data, the raw hyperspectral image sensor data, and the raw digital picture data; the raw hyperspectral image sensor data, the raw laser speckle image data, the raw digital picture data, and 3D surface image data; the raw hyperspectral image sensor data, the raw laser speckle image data, 3D surface image data, and the raw digital picture data; the raw hyperspectral image sensor data, 3D surface image data, the raw laser speckle image data, and the raw digital picture data; 3D surface image data, the raw hyperspectral image sensor data, the raw laser speckle image data, and the raw digital picture data; the raw digital picture data, the raw hyperspectral image sensor data, the raw laser speckle image data, and 3D surface image data; the raw digital picture data, the raw hyperspectral image sensor data, 3D surface image data, and the raw laser speckle image data; the raw digital picture data, 3D surface image data, the raw hyperspectral image sensor data, and the raw laser speckle image data; or 3D surface image data, the raw digital picture data, the raw hyperspectral image sensor data, and the raw laser speckle image data). Furthermore, the following images can be represented/displayed sequentially as described above: the raw, processed or analyzed laser speckle image data; the raw, processed or analyzed hyperspectral image sensor data; the raw, processed or analyzed digital picture data (e.g., an RGB picture); and the 3D surface image. In a particular embodiment, a user may select the particular raw, processed, or analyzed data for viewing via a graphical user interface (GUI), as described in greater detail below.

In an embodiment, the handheld device or system comprises a cradle configured to hold a mobile device (e.g., a tablet or a phone). In a particular embodiment, the cradle is dimensioned such that the mobile device (e.g., a two-in-one computer) is slid into a locked position (i.e., a position that requires a force be applied to move the mobile device from the position). The cradle may comprise a back plate that is substantially the same width as the mobile device, at least one of a side wall (e.g., two side walls) and a bottom wall substantially the same depth as the mobile device. The device or system may further comprise at least two retention pieces that extend from the bottom and/or side walls. The retention pieces can be shaped such that the mobile device can only be slid into the cradle from the top or the bottom of the handheld device. In another embodiment, the rejection pieces can be shaped such that the mobile device, a display screen, and/or a medical robotic apparatus can be snapped into the cradle from the front portion of the cradle. In another embodiment, the cradle is dimensioned such that the mobile device, display screen, and/or the medical robotic apparatus is snapped into place. In an additional embodiment, the retention pieces can be locked into place once the mobile device, the display screen, and/or the medical robotic apparatus is placed in the cradle. For example, the retention pieces can be retained in the cradle via a screw, bolt, a groove and a protrusion, or any other appropriate means. That is, the cradle can contain at least one groove and the retention pieces comprise a protrusion that associates with the grooves of the cradle. A similar groove-protrusion mechanism may be implemented as a mechanism to lock the mobile device, the display screen, and/or the medical robotic apparatus into the cradle. For instance, a protrusion on the cradle may engage a groove on the mobile device, the display screen, or and/or the medical robotic apparatus, or vice versa (i.e., the cradle includes a groove and the attached item includes a protrusion).

In an embodiment, the at least one light source is a light emitting diode (LED) and/or a laser (e.g., a laser diode or a vertical cavity surface emitting laser (VCSEL)). That is, the at least one light source is at least one of a LED and a laser. For example, there can be a plurality of LED and/or a plurality of lasers emitting light in the visible range. Furthermore, the LED can be an LED array and the VCSEL can be a VCSEL array. Laser diodes are semiconductor devices that produce coherent light in the visible or infrared spectrum when current is passed through the diode. In an embodiment, the laser diode is a edge emitting laser diode. In a further embodiment, a VCSEL and/or a plurality of VCSEL will be used as a coherent light source. VCSELs and VCSEL arrays are more compact lasers and produce light with greater uniformity and reduced speckle patterns compared to laser diodes (e.g., edge-emitting laser diodes). In a particular embodiment, VCSELs are used, which are commercially available, and provide wavelengths as low as 630 nm, power levels in excess of 0.5 mwatt, a circular beam shape, low noise, over a modulation bandwidth, and good control of the mode shape and optical beam properties. Such VCSELs are stable and have low values of relative intensity noise (RIN). Power efficiency, small size, and low operating currents of VCSELs (-few mA) minimize the required operating power, and heat dissipation requirements, further improving the suitability of VCSELs attractive for optical imaging applications. VCSELs in particular are relatively cost effective. VCSELs produce a light optical beam with properties that reduce the size burden of additional corrective optical elements. Overall, VCSELs further enable a more compact, energy efficient, processing efficient, coherent light source for optical imaging.

The at least one light source can comprise a wavelength in a range of at least one of: about 400 nm to about 2,500 nm, which can be a LED; and about 600 nm to about 2,500 nm, which can be a laser. In a particular embodiment, the at least one light source emits light at a wavelength in a range of about 630 nm to about 695 nm (e.g., about 688 nm) and about 570 nm to about 630 nm (e.g., about 600 nm), which can be emitted from a LED. In a particular embodiment, a LED emits a visible light with a wavelength of about 570 nm to about 630 nm. In certain embodiments, a laser (e.g., a laser diode or a VCSEL) emits the visible light with a wavelength of about 630 nm to about 805 nm. In an embodiment, the LED is an LED array that emits visible light and/or near infrared light, as described above. In another embodiment, each wavelength produced by an LED is produced by discrete LEDs. In a particular embedment, the laser is a laser array (e.g., a VCSEL array) that emits visible light and/or near infrared light, as described above. In an embodiment, each wavelength produced by a VCSEL is produced by discrete VCSELs.

In an embodiment, the at least one light source emits light at a wavelength in a range of at least one of: about 770 nm to about 815 nm (e.g., about 780 nm to about 805 nm) and about 850 nm to about 2,500 nm (e.g., about 865 nm to about 1,500 nm). In a particular embodiment, at least one LED (e.g., a LED array) emits a near infrared light with a wavelength of about 770 nm to about 815 nm and/or about 850 nm to about 2,500 nm.

In a particular embodiment, the at least one light source is at least one laser or laser array (e.g., a VCSEL or a VCSEL array) emitting light at about 630 nm to about 695 nm (e.g., about 688 nm) and/or about 780 nm to about 805 nm (e.g., about 795 nm). In another embodiment, the at least one light source is at least one LED or an LED array emitting light at about 570 nm to about 630 nm (e.g., about 600 nm), about 770 nm to about 815 nm (e.g., about 780 nm to about 805 nm), and/or about 850 nm to about 2,500 nm (e.g., about 870 nm to about 1,500 nm).

In certain embodiment, the at least one light source (e.g., a laser(s) and/or a LED(s)) emit light in a pulse manner. That is, in an embodiment, the laser(s) and/or LED(s) are repeatedly activated and deactivated during imaging. For example, the at least one light source has a pulse rate of about one pulse of light every about 1 nanosecond to about 3 seconds (e.g., about 500 nanoseconds, about 1000 nanoseconds, about 1500 nanoseconds, about 2000 nanoseconds, about 2500 nanoseconds, about 3000 nanoseconds, about 3500 nanoseconds, about 4000 nanoseconds, about 4500 nanoseconds, about 5000 nanoseconds, about 5500 nanoseconds, about 6000 nanoseconds, about 6500 nanoseconds, about 7000 nanoseconds, about 7500 nanoseconds, about 8000 nanoseconds, about 8500 nanoseconds, about 9000 nanoseconds, about 9500 nanoseconds, about 0.01 milliseconds, about 0.1 milliseconds, about 0.2 milliseconds, about 0.3 milliseconds, about 0.4 milliseconds, about 0.5 milliseconds, about 0.6 milliseconds, about 0.7 milliseconds, about 0.8 milliseconds, about 0.9 milliseconds, about 1 milliseconds, about 100 milliseconds, about 200 milliseconds, about 300 milliseconds, about 400 milliseconds, about 500 milliseconds, about 600 milliseconds, about 700 milliseconds, about 800 milliseconds, about 900 milliseconds, about 1 second, about 1.25 seconds, about 1.5 seconds, about 1.75 seconds, about 2 seconds, about 2.25 seconds, about 2.5 seconds, or about 2.75 seconds) with a duration of light ranging from about 0.5 nanoseconds to about 1 second (e.g., about 500 nanoseconds, about 1000 nanoseconds, about 1500 nanoseconds, about 2000 nanoseconds, about 2500 nanoseconds, about 3000 nanoseconds, about 3500 nanoseconds, about 4000 nanoseconds, about 4500 nanoseconds, about 5000 nanoseconds, about 5500 nanoseconds, about 6000 nanoseconds, about 6500 nanoseconds, about 7000 nanoseconds, about 7500 nanoseconds, about 8000 nanoseconds, about 8500 nanoseconds, about 9000 nanoseconds, about 9500 nanoseconds, about 0.01 milliseconds, about 0.1 milliseconds, about 0.2 milliseconds, about 0.3 milliseconds, about 0.4 milliseconds, about 0.5 milliseconds, about 0.6 milliseconds, about 0.7 milliseconds, about 0.8 milliseconds, about 0.9 milliseconds, about 1 milliseconds, about 100 milliseconds, about 200 milliseconds, about 300 milliseconds, about 400 milliseconds, about 500 milliseconds, about 600 milliseconds, about 700 milliseconds, about 800 milliseconds, or about 900 milliseconds). This control and the combination of LED and VCSEL technologies provides for a more energy platform, which is particularly important in resource constrained platforms such as, mobile, portable, handheld, and/or battery operated devices, and more efficient relative to the use of halogen lamps or fiberoptic light sources, as seen in some imaging technologies. As a result, the use of LED(s) and/or laser(s) (e.g., laser diode or VCSEL) provides markedly more energy efficient light sources for the source assembly.

In other embodiments, the at least one light source emits light in a continuous manner, for example, for about 0.5 seconds to about 60 seconds. In a particular embodiment, the at least one light source (e.g., a LED or VCSEL) emits light for about 0.5 second to about 50 seconds, about 0.5 seconds to about 40 seconds, about 0.5 seconds to about 35 seconds, about 0.5 seconds to about 30 seconds, about 0.5 seconds to about 25 seconds, about 0.5 seconds to about 20 seconds, about 0.5 seconds to about 15 seconds, about 0.5 seconds to about 10 seconds, about 0.5 seconds to about 5 seconds, about 1 to about 60 seconds, about 1 second to about 50 seconds, about 1 seconds to about 40 seconds, about 1 seconds to about 35 seconds, about 1 seconds to about 30 seconds, about 1 seconds to about 25 seconds, about 1 seconds to about 20 seconds, about 1 seconds to about 15 seconds, about 1 seconds to about 10 seconds, about 1 seconds to about 5 seconds, about 3 to about 60 seconds, about 3 second to about 50 seconds, about 3 seconds to about 40 seconds, about 3 seconds to about 35 seconds, about 3 seconds to about 30 seconds, about 3 seconds to about 25 seconds, about 3 seconds to about 20 seconds, about 3 seconds to about 15 seconds, about 3 seconds to about 10 seconds, about 3 seconds to about 5 seconds, about 5 to about 60 seconds, about 5 second to about 50 seconds, about 5 seconds to about 40 seconds, about 5 seconds to about 35 seconds, about 5 seconds to about 30 seconds, about 5 seconds to about 25 seconds, about 5 seconds to about 20 seconds, about 5 seconds to about 15 seconds, about 5 seconds to about 10 seconds, about 5 seconds to about 8 seconds, about 8 to about 60 seconds, about 8 second to about 50 seconds, about 8 seconds to about 40 seconds, about 8 seconds to about 35 seconds, about 8 seconds to about 30 seconds, about 8 seconds to about 25 seconds, about 8 seconds to about 20 seconds, about 8 seconds to about 15 seconds, about 8 seconds to about 10 seconds, about 10 to about 60 seconds, about 10 second to about 50 seconds, about 10 seconds to about 40 seconds, about 10 seconds to about 35 seconds, about 10 seconds to about 30 seconds, about 10 seconds to about 25 seconds, about 10 seconds to about 20 seconds, about 10 seconds to about 15 seconds, about 10 seconds to about 13 seconds, about 13 to about 60 seconds, about 13 second to about 50 seconds, about 13 seconds to about 40 seconds, about 13 seconds to about 35 seconds, about 13 seconds to about 30 seconds, about 13 seconds to about 25 seconds, about 13 seconds to about 20 seconds, about 13 seconds to about 15 seconds, about 15 to about 60 seconds, about 15 second to about 50 seconds, about 15 seconds to about 40 seconds, about 15 seconds to about 35 seconds, about 15 seconds to about 30 seconds, about 15 seconds to about 25 seconds, about 15 seconds to about 20 seconds, about 15 seconds to about 18 seconds, about 18 to about 60 seconds, about 18 second to about 50 seconds, about 18 seconds to about 40 seconds, about 18 seconds to about 35 seconds, about 18 seconds to about 30 seconds, about 18 seconds to about 25 seconds, about 18 seconds to about 20 seconds, about 20 to about 60 seconds, about 20 second to about 50 seconds, about 20 seconds to about 40 seconds, about 20 seconds to about 35 seconds, about 20 seconds to about 30 seconds, about 20 seconds to about 25 seconds, about 25 to about 60 seconds, about 25 second to about 50 seconds, about 25 seconds to about 40 seconds, about 25 seconds to about 35 seconds, about 25 seconds to about 30 seconds, about 30 to about 60 seconds, about 30 second to about 50 seconds, about 30 seconds to about 40 seconds, about 30 seconds to about 35 seconds, about 35 to about 60 seconds, about 35 second to about 50 seconds, about 35 seconds to about 40 seconds, about 40 to about 60 seconds, about 40 second to about 50 seconds, about 40 seconds to about 45 seconds, about 45 seconds to about 60 seconds, about 45 to about 50 seconds, about 50 second to about 60 seconds, or about 55 seconds to about 60 seconds.

In particular embodiments, a diffuser lens (e.g., a micro-diffuse lens) is located in a light path of the at least one light source located between the at least one light source and the region of interest. In an embodiment, the light path is produced by at least one laser, such as a VSCEL or VCSEL array. The diffuser lens can have a divergence angle of about 5 to about 50 degrees depending upon the distance of the device from the region of interest. For example, the divergence angle of the diffuser lens in a range of about 5 degrees to about 50 degrees, about 5 degrees to about 45 degrees, about 5 degrees to about 40 degrees, about 5 degrees to about 35 degrees, about 5 degrees to about 30 degrees, about 5 degrees to about 25 degrees, about 5 degrees to about 20 degrees, about 5 degrees to about 15 degrees, about 5 degrees to about 10 degrees, about 10 degrees to about 50 degrees, about 10 degrees to about 45 degrees, about 10 degrees to about 40 degrees, about 10 degrees to about 35 degrees, about 10 degrees to about 30 degrees, about 10 degrees to about 25 degrees, about 10 degrees to about 20 degrees, about 10 degrees to about 15 degrees, about 15 degrees to about 50 degrees, about 15 degrees to about 45 degrees, about 15 to about 40 degrees, about 15 degrees to about 35 degrees, about 15 degrees to about 30 degrees, about 15 degrees to about 25 degrees, about 15 degrees to about 20 degrees, about 20 degrees to about 50 degrees, about 20 degrees to about 45 degrees, about 20 to about 40 degrees, about 20 degrees to about 35 degrees, about 20 degrees to about 30 degrees, about 20 degrees to about 25 degrees, about 25 degrees to about 50 degrees, about 25 degrees to about 45 degrees, about 25 to about 40 degrees, about 25 degrees to about 35 degrees, about 25 degrees to about 30 degrees, about 35 degrees to about 50 degrees, about 35 degrees to about 45 degrees, about 30 degrees to about 40 degrees, about 30 degrees to about 35 degrees, about 35 degrees to about 50 degrees, about 35 degrees to about 45 degrees, about 35 degrees to about 40 degrees, about 40 degrees to about 50 degrees, about 40 degrees to about 45 degrees, or about 45 degrees to about 50 degrees.

In another embodiment, a polarizing film and/or lens is located in the light path of the at least one light source (e.g., at least one LED or an LED array). For example, the polarizing film and/or lens can be located within the path of light from the at least one light source to the region of interest, wherein the at least one light source emits light with a wavelength in the visible and near infrared spectrum, e.g., about 600 nm to about 900 nm.

In another embodiment, a polarizing film and/or lens located in the light path will polarize light in a number of directions including, circular and/or linear, and/or elliptical. In another embodiment, a polarizing film and/or lens located in the light path will polarize light by some combination of directions and/or some combination of rotations (e.g., R/S for linear and/or clockwise/counterclockwise for circular), wherein at least one form and type of polarizing element is located in the light path.

Medical hyperspectral imaging (HSI) is an imaging modality utilized by the medical field to provide diagnostic information about tissue physiology, morphology, and composition. Hyperspectral imagers acquire three-dimensional datasets, referred to as a hypercube. Light absorbed by tissue constituents is converted to heat or radiated in the form of luminescence, e.g., fluorescence and phosphorescence, which is captured by the hyperspectral imager. Medical HSI is successful because it is complete, using the spectral data of reflected electromagnetic radiation (ultraviolet—UV, visible, near infrared—NIR, and infrared—IR), and since different types of tissue reflect, absorb, and scatter light differently, in theory the hyperspectral cubes contains enough information to differentiate between tissue types and conditions. The hypercube has two spatial dimensions (x, y) and one spectral dimension (λ)—that is, a range of wavelengths. The spatially resolved spectral image can be utilized for diagnostic purposes. Medical HSI is robust since it is based on a few general properties of the spectral profiles (slope, offset, and ratio) therefore is flexible with respect to spectral coverage. Medical HSI uses fast image processing techniques that allow superposition of absorbance, scattering, and oxygenation information in one pseudo-color image. For example, the concentration and oxygen saturation of hemoglobin can be determined via its absorption spectra. This can be utilized to detect angiogenesis and hypermetabolism, both of which are associated with cancer. Similarly, one can distinguish between various types of tissues, e.g. epithelial and connective tissue because collagen or elastin excited at a wavelength in the range of about 300 nm to about 400 nm has broad emission bands between about 400 nm and about 600 nm. Furthermore, cells in different disease states exhibit different structures or different metabolism rates, which result in detectable differences within their fluorescence emission spectra. Similarly, changes in disease states result in corresponding changes in the patterns of light reflected from the tissue, which can be detected within the emission spectra. Medical HSI can deliver results in a very intuitive form by pairing a medical HSI pseudo-color image with the high quality color picture composed from the same hyperspectral data. Additional information regarding hyperspectral imaging can be found in, for example, Guolan Lu and Baowei Fei (Medical hyperspectral imaging: a review. Journal of Biomedical Optics 19(1): 010901 (January 2014)) and U.S. Pat. No. 8,655,433 B2 both of which are incorporated herein by reference.

In an embodiment, the device comprises an optic responsive to illumination of a tissue, a spectral separator, an imaging sensor, a heterogeneous processor configuration, a filter control interface, and a general-purpose operating module. In an embodiment, the spectral separator is optically responsive to the optic and has a control input. In an embodiment, the device further comprises a polarizer that compiles a plurality of light beams into a plane of polarization before entering the imaging sensor. The imaging sensor can be optically responsive to the spectral separator. In another embodiment, the heterogeneous processor comprises an image acquisition interface with an input responsive to the imaging sensor and one or more diagnostic protocol modules. Each protocol can comprise a set of instructions for operating the spectral separator and for operating the filter control interface. The filter control interface can comprise a control output provided to the control input of the spectral separator, which directs the spectral separator independently of the illumination to receive one or more wavelengths of the illumination to provide multispectral or hyperspectral information as determined by the set of instructions provided by the one or more protocol module, and the general-purpose operating) module performs filtering and acquiring steps one or more times depending on the set of instructions provided by the one or more protocol modules. In another embodiment, the multispectral and/or hyperspectral information determines one or more of: presence of tissue pathology for screening or diagnosis; presence of signs of disease; and progression of tissue healing/regeneration. The images can be seen on a mobile display, computer screen, display screen or projector, and/or stored and transported as any other digital information, and/or printed out.

In an embodiment, the hyperspectral image sensor detects wavelengths in the visible light range and near infrared light, i.e., wavelengths in a range of 400 nm to 2,500 nm (e.g., about 400 nm to about 1,200 nm or about 600 nm to about 900 nm). In a certain embodiment, the hyperspectral image sensor captures about 90 to about 180 frames per second. In a particular embodiment, the hyperspectral image sensor takes images for about 1 second to about 20 seconds. In an embodiment, the hyperspectral image sensor takes images for about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, or about 19 seconds. For example, the hyperspectral image sensor can take about 30 to about 180 frames per second (e.g., about 90 to about 120 frames per second) for about 1 second to about 20 seconds (e.g., about 5 seconds). The hyperspectral image sensor can be a snapshot hyperspectral imager, i.e., the imager does not require scanning, but rather takes discrete images. Discrete images taken at these rates avoid motion artifacts, simplifying data processing, thereby decreasing processing time and energy requirements. In an embodiment, the hyperspectral image sensor is selected from the group consisting of a camera, a charge-coupled device (CCD) camera/image sensor and a complementary metal-oxide-semiconductor (CMOS) camera/image sensor. Furthermore, the hyperspectral image sensor can be a hyperspectral snapshot image sensor. Hyperspectral snapshot imagers provide substantially equivalent spatial resolution, high temporal resolution, and are relatively faster at acquiring images and are more energy efficient than scanning hyperspectral imagers due to the reduction in image data processing, the ‘snapshot’ capture mode and targeted analysis of less than 100 spectral bands.

A snapshot multispectral imager may allow for an entire image to be captured at one discrete point in time, which can be realized by adding three key steps to the use of monolithically integrated filters: (1) organizing the filters in a tiled configuration, where each filter is designed to sense only one narrow band of wavelengths; (2) adding an optical subsystem that duplicates the scene onto each filter tile; and (3) using an objective lens that forms an image of the scene on the optical duplicator. A set of FP filters can be made by traditional semiconductor fabrication tools, enabling mass production and, therefore, low cost manufacturing. The distinct filters may cover part of the VNIR (visible and near-infrared) range for which CMOS imagers are sensitive (e.g., 600-1500 nm). Each tile senses one selected wavelength by varying the cavity length for each filter. This is achieved by using a set of CMOS-compatible production steps such as deposition, patterning and etching. The optical duplicator subsystem can be made of a microlens array and optomechanical components for assembly. The duplicator relays the light of the objective lens onto the sensor through multiple optical channels, each consisting of one lens or lens system. A hyperspectral snapshot camera may comprise a replaceable objective lens at the front and duplication optics between the objective and the camera, which may relay and copy the area of interest as seen through the objective lens onto the tiled filters of the sensor inside the camera. A hyperspectral snapshot technology functions to divide light spectrum into many small wavelength bands in the Visible and Near-IR at a small bandwidth per band, along with high spatial resolution, and the ability to capture up to 180 hyperspectral cubes per second. A hyperspectral snapshot imager can reduce the burden of processing large amounts of spectral data as is encountered with in line-scan hyperspectral imagers, while maintaining low cost, flexible, high speed imaging, and compact design. Hyperspectral snapshot imagers also provide opportunities in video-rate (i.e., a video recording) and non-scanning application as well. For example, the hyperspectral snapshot imager can be utilized to perform spectral imaging, laser speckle imaging, or both spectral imaging and laser speckle imaging. The hyperspectral snapshot imager can also be used to take a video recording. The design and relative lower cost of hyperspectral snapshot imager allow for a more flexible solution than typical hyperspectral imagers. As such, a hyperspectral snapshot imager allows for application-specific customizations and reiterations of wafer designs and expanded spectral coverages.

In another embodiment, the imaging device and/or system acquires, extracts, and interprets up to 25 spectral bands within wavelengths ranging from about 400 nm to about 900 nm. For example, each spectral band can be adjacent to another, thereby forming a substantially continuous set of spectral bands. In an embodiment, the device/system acquires, extracts, and interprets ≦20 spectral bands, ≦15 spectral bands, ≦10 spectral bands, or ≦5 spectral bands. For example, the device/system can acquire, extract, and interpret 1, 2, 3, 4, 5, 6, 7, 8, 9, 10, 11, 12, 13, 14, 15, 16, 17, 18, 19, 20, 21, 22, 23, 24, or 25 spectral bands. In certain embodiments, each spectral band has a bandwidth of ≦ about 50 nm, e.g., ≦ about 30 nm or ≦ about 20 nm. In a particular embodiment, each spectral band has a wavelength in a range of about 20 nm to about 40 nm, about 20 nm to about 30 nm, about 10 nm to about 2.0 nm, about 10 nm to about 15 nm, or about 10 nm to about 12 nm. In an embodiment, each spectral band has a bandwidth ≦ about 15 nm. For example, in an embodiment, the hyperspectral imager captures light in at least one of the following (e.g., all of the following) wavelengths: about 605 nm, about 617 nm, about 629 nm, about 641 nm, about 653 nm, about 665 nm, about 677 nm, about 689 nm, about 701 nm, about 713 nm, about 725 nm, about 737 nm, about 749 nm, about 761 nm, about 773 nm, about 785 nm, about 797 nm, about 809 nm, about 821 nm, about 833 nm, about 845 nm, about 857 nm, about 869 nm, about 881 nm, and about 895 nm. In an embodiment, the region of interest is illuminated by at least one LED (e.g., a plurality of LEDs) or a LED array, which can emit light at a wavelength in a range of about 400 nm to about 2,500 nm. The responsiveness to illumination of the tissue with one or more wavelengths of light in at least one of visible, NIR, and IR light is utilized in the production of a hyperspectral image.

Laser speckle imaging is an imaging modality utilized by the medical field to measure, e.g., the superficial blood flow in tissue. Laser Speckle Contrast Imaging (LSI/LSCI) and variations thereof, can serve to further investigate microvasculature, tissue oxygenation, distinct blood particulates, and in the computational 3D reconstruction of structures related to the observed anatomy. LSCI is a minimally invasive method used to image blood flow by illuminating chromophores hemoglobin) within the blood in vivo and tracking their relative velocities in the blood as they flow with high spatial and temporal resolution by utilizing the interference effects of a coherent light source. Non-moving scattering particles in the media produce a stable speckle pattern, whereas movement of scattering particles causes phase shifts in the scattered light, and temporal changes in the speckle pattern. In LSCI, time integrated speckle pattern can be used to estimate blood flow n a tissue. Combining the blood flow oxygenation information can provide a better estimate of underlying soft tissue activity and serve as an indirect indicator of metabolic dynamics. Monochromatic light from a laser source is directed to the region of interest. As an example, the light scattered within an area of interest can be analyzed as a product of blood cells moving through and by adjacent stationary structures (also referred to as backscatter) to determine, e.g., the number of blood cells, the average velocity of the bloods cells, and microvascular structures (e.g., stationary structures). Additional information regarding laser speckle imaging can be found in, for example, U.S. Pat. No. 4,590,948, U.S. Pat. No. 6,045,511, and International Patent Application Publication WO 2011/029086, each of which are incorporated herein by reference.

In an embodiment, the laser speckle image data is acquired by the camera of the mobile device, the handheld device, or the system, as described herein. Alternatively, the laser speckle image data is acquired by the hyperspectral snapshot imager. In an additional embodiment, the camera or hyperspectral snapshot imager detects the backscatter of the region of interest (i.e., the laser speckle image data) from coherent light with a wavelength in the range of at least one of about 630 nm to about 695 nm (e.g., about 688 nm) and about 770 nm to about 815 nm (e.g., about 795 nm).

In an embodiment, this optical imaging system is operable to alternate the operation of at least one multi-modal, multi-wavelength light source between at least two different modalities, such as laser speckle contrast imaging (LSCI) and medical hyperspectral imaging, thereby enabling the reduction of spatial noise and temporal noise and enhanced visible and NIR spectral analysis in resulting image data. As another aspect of this embodiment, a novel optical imaging scheme for use in conjunction with laser light sources is provided, that includes alternating the light source(s) between a single mode and a multi-modal, continuous or pulsed, and optionally multi-wavelength sweep modes of the area of interest, thereby enabling the manipulation of speckle and spectral noise properties and/or corrections. For example, the multi-wavelength sweep mode produces light from a lower wavelength to a higher wavelength within a range or from a higher wavelength to a lower wavelength.

In an embodiment, the coherent light source for LSCI is at least one VCSEL, which can optionally be used for depth sensing. VCSELs are commercially available, and provide wavelengths as low as 670 nm, power levels in excess of 0.5 mwatt, a circular beam shape, low noise, over a GHz modulation bandwidth, and good control of the mode shape and optical beam properties. Such VCSELs are stable and have low values of relative intensity noise (RIN). Power efficiency, small size, and low operating currents of VCSELs (a few mA) minimize the required operating power, and heat dissipation requirements, further improving the suitability of VCSELs for optical imaging applications. VCSELs in particular are relatively cost effective. VCSELs produce a more uniform light optical beam properties (i.e., spherical) thereby reducing the size burden of additional corrective optical elements. Overall, VCSELs further enable a more compact, energy efficient, processing efficient, coherent light source for imaging.

In an embodiment, the device, control module, and system is operable to alternate the operation of a laser light source between a single illumination mode (or pattern), continuous or pulsed, and optionally can operate a sweep mode illumination (or pattern), thereby enabling the manipulation of speckle noise properties or coherence effects of captured images, and can leverage such modes with any number of sensors including, a hyperspectral image sensor and a camera, an IR image sensor and camera, a CMOS type image sensor, a CCD type image sensors, and/or depth sensors. In an embodiment, the sweep mode illumination can move across the region of interest or produce a particular pattern of light on the region of interest. In this particular aspect of the invention, the laser light source is a multi-modal, multi-wavelength VCSEL, and the device or system of the present invention is operable to alternate the operation of the VCSEL between modes. It should be noted that any light source that becomes available that have a more compact structure and improved beam uniformity may also be suited for use in the present invention.

In an embodiment, 3D surface imaging is used to measure depth and dimensions of the region of interest. The 3D surface image surface data can be utilized to produce a virtual image of the area of interest. 3D surface imaging can be achieved through a combination of light, depth sensors, and image detectors. The image data can be analyzed and used to reconstruct 3D surface images of the objects, which is slightly more superficial than the reconstructions achieved by LSCI (e.g., micrometers to millimeters). The accessibility of depth sensing technologies, cameras, and software in consumer electronic devices such as, a select group of mobile and gaming devices, has presented an opportunity to leverage 3D imaging techniques and technologies to visualize tissue disease and architecture in a more interactive way. Independent of other clinical information, additional and reliable image sensors and data, this modality does not provide sufficient specificity and/or does not achieve sufficient depth to diagnose tissue disease. However, the ability to leverage 3D surface imaging in the context of other imaging modalities (digital, spectral, and/or speckle imaging) would be advantageous for the assessment, monitoring, diagnosis, and management of tissue diseases. In combination with other modalities that are able to evaluate blood flow, tissue oxygenation, tissue health, and other anatomical and physiological signs, can serve to further evaluate dimensions of disease and supplement 3D visual aids to caregivers and providers by way of telemedicine portals, video-based simulations for intervention planning, and an aggregation of dimensions data over-time for structural monitoring related to tissue disease and tissue healing and regeneration.

In a particular embodiment, the thermographic sensor and/or processing is capable of differentiating a heat signature of an immune response (e.g., an inflammatory immune response) as compared to evaluating tissue pathology and monitoring/evaluating tissue healing and regeneration. Thermographic imagers may be used to evaluate soft tissue characteristics such as, inflammation, infection (non-specific), and blood flow. Thermographic sensors detect radiation emitted by objects above absolute zero. The radiation emitted by objects has a wavelength in a range of about 9,000 nm to about 14,000 nm. Thermal images (or thermograms) are visual displays of the amount of infrared energy emitted, transmitted, and reflected by an object. Warmer objects emit greater amounts of emitted radiant energy. Thermographic sensors detect energy emitted, transmitted and reflected by an object and through processing produces images that illustrate the relative difference in the temperature within the imaged region. That is, the thermogram illustrates to a user areas of the image which are warmer or colder than others. In an alternative embodiment, the handheld device or system comprises a thermographic sensor. Independent of any other clinical data, imaging data, or imaging technology the modality of thermal image data does not provide sufficiently reliable and specific data to significantly impact clinical care related to tissue diseases, except to identify tissue ‘cold’ spots that might supplement investigations of blood flow. As a combination of a larger suite of imaging technologies, however, thermographic imaging can improve the precision and reliability of findings that relate to blood flow, inflammation, infection, and other physiological processes.

In an embodiment, the multi-modal imaging device is achieved through a combination of board level configurations (e.g., heterogeneous computation board) and interface components (e.g., SuperSpeed or SuperSpeed+ processing/transfer mode).

In an embodiment, the hyperspectral image sensor comprises a high speed camera that transfers data at up to 5 Gbit/s (625 MB/s) (e.g., using the SuperSpeed transfer mode/process utilized by the USB 3.0 standard). A USB 3.0 compliant processing cameras maintains SuperSpeed USB compliant processing and therefore, data transfer rates. The hyperspectral image sensor can be a snapshot mosaic hyperspectral image sensor. In another embodiment, the hyperspectral image sensors transfers data at up to 10 Gbits/s (1.25 GB/s) (e.g., using the SuperSpeed+ transfer mode/process utilized by the USB 3.1 standard).

The device can also include a data port or plug, e.g., a USB 3.0 or 3.1 compliant data port/plug. The data port or plug can be utilized for peripheral imagers. The device or system's ability to perform SuperSpeed and SuperSpeed+ processing/data transfers provide the ability to run faster and/or higher resolution image sensors. Increased bandwidth allows more data to be transmitted, such as full color processed images (e.g., Point Grey Inc. USB 3.0 cameras), which removes the requirement for the host system to perform this processing. As a result, CPU resource usage is less for USB 3.0 or 3.1 based camera systems than with other processing and data transfer modes, e.g., USB 2.0, GigE, and FireWire. Furthermore, the USB 3.0/3.1 peripheral sensors and SuperSpeed/SuperSpeed+ cameras allow for a number of devices to be connected to the same host, which is advantageous for multi-modal imaging devices. USB 3.0/3.1 peripheral sensors and cameras also improve energy efficiency due to asynchronous signaling, rather than constant polling, which allows a device to notify the host when it is ready for data transfer, and a variety of other protocol improvements, such as streaming support for bulk transfers and a more efficient token/data/handshake sequence, improve system efficiency and reduced power consumption, and offer an improved mechanism for entering and exiting low-power states, which is advantageous in resource constrained applications such as, battery operated mobile, portable medical robotic, and handheld devices.

In an embodiment, the control module includes at least one of a heterogeneous computational board and microcontrollers. In a particular embodiment, the heterogeneous computational board comprises at least one of a field-programmable gate array (FPGA), a graphic processing unit (GPU), and a central processing unit (CPU). A FPGA is an integrated circuit that can be configured after manufacture, for example, using a hardware description language (HDL). FPGAs are capable of implementing complex digital computations. The FPGA can comprise memory, e.g. non-transitory computer readable medium. The ability of FPGAs to perform parallel computations results in FGPAs being significantly faster for some applications, e.g. computer vision applications. CPUs are electronic circuitry that can carry out the instructions of a program stored in computer memory, e.g. non-transitory computer readable medium. GPUs are a specialized electronic circuit designed to rapidly manipulate and alter memory to accelerate the creation of images for output to a display. GPUs can also be used for general-purpose computing, as opposed to being hard wired to only perform graphical operations. The highly parallel structure of GPUs make them more efficient than CPUs for algorithms where processing large blocks of data is done in parallel. For example, some computations can be performed up to forty times faster by a GPU as compared to a conventional CPU. In some embodiments, the heterogeneous computation board is configured to process image data from multiple sources. The heterogeneous computational board can be configured to rapidly evaluate image data from multiple sensors, imagers, and/or cameras from an area of interest to provide real-time analysis of tissue pathology and tissue healing, to monitor tissue regeneration, and/or to monitor tissue disease progression.

The heterogeneous computational board can be configured to process the hyperspectral image data. Analyzing the hyperspectral image data with the FPGA, the GPU, and the CPU allows for the device or system to be optimized for reduced power consumption as compared to traditional hyperspectral processing systems. In particular, the heterogeneous computational board allows for each portion of the hyperspectral image data processing to be performed by the processor (e.g. FPGA, GPU, or CPU) that has the highest efficiency for each task. That is, power consumption can be reduced by excising algorithms required when using a pure GPU based solution, a pure FPGA based solution or a combination of FPGA and GPU based solutions, and can be performed more efficiently that if it were performed by only a CPU. For example, FPGA and/or GPU based algorithms can analyze the hyperspectral image data faster and more efficiently than CPU based solutions.

In a certain embodiment, the scatter data, e.g., the laser speckle image data acquired by the camera or hyperspectral image sensor, is processed by the FPGA, the GPU, or the FPGA and the GPU. FPGA and/or GPU processing of scatter data is more efficient than processing the scatter data strictly by the CPU. In certain embodiments, the FPGA performs a pure pixel algorithm for processing the scatter data, which is a parameterizable HDL based solution. The parameterizable HDL based solution can be a parallelizing of the mathematically intensive algorithms presently utilized in processing laser speckle backscatter data/light. The parameterizable HDL based solution can increase data throughput of the image, improve battery life, and can be adapted to different FPGAs. Similarly, the GPU can be used to perform the pure pixel algorithm for processing the scatter data in a parallelization of the mathematical intensive algorithms presently utilized in processing laser speckle backscatter data/light. In a certain embodiment, the CPU, GPU and/or FPGA capabilities, and components of other devices (e.g., mobile device(s), robotic device(s), handheld device(s)) may also be leveraged to further reduce the energy consumption and processing load of the device and system, thereby improving data acquisition, transfer, and analysis rates.

The ability for the FPGA to be reconfigured after manufacture through the use of a HDL allows for additional imaging modalities to be utilized with the device or system after manufacture. For example, a thermographic sensor may be connected to the device or system after manufacture and the FPGA configured to analyze the data produced therefrom. The connection from the sensor and the device or system can be accomplished through a direct connection (e.g., a plug and port) or a wireless connection (e.g., BlueTooth, WiFi, or ZigBee™) or any other similar communication means known in the art. Therefore, in an embodiment, the handheld device or system further comprises a data port or plug. The data port or plug allows for the connection of another electronic device, e.g., another imager or a mobile device, or for charging the device. For example, in an embodiment, a thermographic sensor can be connected to the data port or plug of the device or system.

In a particular embodiment, the FPGA receives the spectral images from the camera and temporarily stores the data in its memory as the FPGA processes the data. In an embodiment, the FPGA is optimized for parallelization of processing and numerous low level system architecture choices. For example, the FPGA can be split into three parts: a skewer manager, a spectral unmixer, and an output pixel manager. In certain embodiments, the skewer manager: a user selects the skewers of interest, which contain information relevant to specific tissue structure, pathology or disease; the skewers can be changed during the analysis process; and multiple skewers can be processed at once, which can be useful for determining if a set of materials are present within the image. The spectral unmixer can perform the spectral unmixing by applying the skewer, which may have been acquire by the skewer manager, to the image by use of a dot product across all spectral values. In a particular embodiment, the output pixel manager assembles the processed image and outputs this to the memory through a data connection, which can be displayed on the mobile device, the display of the device, or the display of a remote device. In another embodiment, the FPGA only reads the original hyperspectral image from the memory and stores the output image to the memory. For example, the FPGA can utilize local block ram within the FPGA to store intermediate data of the processing of the data (e.g., hyperspectral data). In an embodiment, each 5×5 spectral image block containing data for various spectral wavelengths are unmixed/processed a line at a time. This is discussed in greater detail below. The processed data is stored in an intermediate data store (e.g., local block ram) while the remaining lines are processed. As each pixel (i.e., 5×5 spectral image block) is assembled, the data can be stored in memory (e.g., DDR3 memory), be displayed as described in the present disclosure, or sent to a remote server or computer for storage, display, or further analysis.

In another embodiment, the control module is at least one processor, at least one heterogeneous computational board, and/or at least one microcontroller. For example, in an embodiment, the control module includes a heterogeneous computational board and a processor separate from the heterogeneous computational board or a microcontroller. A microcontroller is a small computer on a single integrated circuit comprising a processor core, memory, and programmable input/output peripherals. In an embodiment, the processor(s), the processor of the microcontroller(s), and the processor of the heterogeneous computational board(s) comprise non-transitory computer readable medium configured to execute instructions for analyzing data from the control module to detect or assess a tissue wound.

In another embodiment, the device or system further comprises at least one power source, e.g., a battery. The battery can be a rechargeable battery, e.g., a lithium rechargeable battery, including a lithium ion battery or a lithium iron phosphate battery, or any other similar power source known in the art. In an embodiment, the device or system has a battery for the control module, e.g. the heterogeneous computational board, and at least one battery for the source assembly and the plurality of acquisition devices, which can include a camera. In another embodiment, the device or system has a battery (for use to power, e.g., the control module and sensors/imagers/cameras contained therein) and is configured to utilize the power supply of the mobile device through a port/plug connection. In contrast to prior imaging technologies, the efficient processing (see heterogeneous computational board above), imaging techniques (see hyperspectral imaging above) and use of light sources (see LED and laser light sources above) allow for the device or system of the present disclosure to energy efficient enough to effectively use a battery as a power source. Previous imaging devices would consume at least 50% of a battery with a single scan, that is, a round of imaging. The device and system of the present disclosure provide an energy efficiency that allows for the device or system to acquire about 5 to about 60 scans, as substantially described herein. In another embodiment, the device or system can perform about 5 to about 50 scans, about 5 to about 40 scans, about 5 to about 30 scans, about 5 to about 25 scans, about 5 to about 20 scans, about 5 to about 15 scans, about 5 to about 10 scans, about 8 to about 60 scans, about 8 to about 50 scans, about 8 to about 40 scans, about 8 to about 30 scans, about 8 to about 25 scans, about 8 to about 20 scans, about 8 to about 15 scans, about 8 to about 10 scans, about 10 to about 60 scans, about 10 to about 50 scans, about 10 to about 40 scans, about 10 to about 30 scans, about 10 to about 25 scans, about 10 to about 20 scans, about 10 to about 15 scans, about 12 to about 60 scans, about 12 to about 50 scans, about 12 to about 40 scans, about 12 to about 30 scans, about 12 to about 25 scans, about 12 to about 20 scans, about 12 to about 15 scans, about 15 to about 60 scans, about 15 to about 50 scans, about 15 to about 40 scans, about 15 to about 30 scans, about 15 to about 25 scans, about 15 to about 20 scans, about 20 to about 60 scans, about 20 to about 50 scans, about 20 to about 40 scans, about 20 to about 30 scans, about 20 to about 25 scans, about 25 to about 60 scans, about 25 to about 50 scans, about 25 to about 40 scans, about 25 to about 30 scans, about 30 to about 60 scans, about 30 to about 50 scans, about 30 to about 40 scans, about 40 to about 60 scans, about 40 to about 50 scans, or about 50 to about 60 scans. In an embodiment the device or system has a battery life of up to about 24 hours (e.g., about 1 hour to about 24 hours, about 1 hour to about 23 hours, about 1 hour to about 22 hours, about 1 hour to about 21 hours, about 1 hour to about 20 hours, about 1 hour to about 19 hours, about 1 hour to about 18 hours, about 1 hour to about 17 hours, about 1 hour to about 16 hours, about 1 hour to about 15 hours, about 1 hour to about 14 hours, about 1 hour to about 13 hours, about 1 hour to about 12 hours, about 1 hour to about 11 hours, about 1 hour to about 10 hours, about 1 hour to about 9 hours, about 1 hour to about 8 hours, about 1 hour to about 7 hours, about 1 hour to about 6 hours, about 1 hour to about 5 hours, about 1 hour to about 4 hours, about 1 hour to about 3 hours, about 1 hour to about 2 hours, about 2 hour to about 24 hours, about 2 hour to about 23 hours, about 2 hour to about 22 hours, about 2 hour to about 21 hours, about 2 hour to about 20 hours, about 2 hour to about 19 hours, about 2 hour to about 18 hours, about 2 hour to about 17 hours, about 2 hour to about 16 hours, about 2 hour to about 15 hours, about 2 hour to about 14 hours, about 2 hour to about 13 hours, about 2 hour to about 12 hours, about 2 hour to about 11 hours, about 2 hour to about 10 hours, about 2 hour to about 9 hours, about 2 hour to about 8 hours, about 2 hour to about 7 hours, about 2 hour to about 6 hours, about 2 hour to about 5 hours, about 2 hour to about 4 hours, about 2 hour to about 3 hours, about 3 hour to about 24 hours, about 3 hour to about 23 hours, about 3 hour to about 22 hours, about 3 hour to about 21 hours, about 3 hour to about 20 hours, about 3 hour to about 19 hours, about 3 hour to about 18 hours, about 3 hour to about 17 hours, about 3 hour to about 16 hours, about 3 hour to about 15 hours, about 3 hour to about 14 hours, about 3 hour to about 13 hours, about 3 hour to about 12 hours, about 3 hour to about 11 hours, about 3 hour to about 10 hours, about 3 hour to about 9 hours, about 3 hour to about 8 hours, about 3 hour to about 7 hours, about 3 hour to about 6 hours, about 3 hour to about 5 hours, about 3 hour to about 4 hours, about 4 hour to about 24 hours, about 4 hour to about 23 hours, about 4 hour to about 22 hours, about 4 hour to about 21 hours, about 4 hour to about 20 hours, about 4 hour to about 19 hours, about 4 hour to about 18 hours, about 4 hour to about 17 hours, about 4 hour to about 16 hours, about 4 hour to about 15 hours, about 4 hour to about 14 hours, about 4 hour to about 13 hours, about 4 hour to about 12 hours, about 4 hour to about 11 hours, about 4 hour to about 10 hours, about 4 hour to about 9 hours, about 4 hour to about 8 hours, about 4 hour to about 7 hours, about 4 hour to about 6 hours, about 4 hour to about 5 hours, about 5 hour to about 24 hours, about 5 hour to about 23 hours, about 5 hour to about 22 hours, about 5 hour to about 21 hours, about 5 hour to about 20 hours, about 5 hour to about 19 hours, about 5 hour to about 18 hours, about 5 hour to about 17 hours, about 5 hour to about 16 hours, about 5 hour to about 15 hours, about 5 hour to about 14 hours, about 5 hour to about 13 hours, about 5 hour to about 12 hours, about 5 hour to about 11 hours, about 5 hour to about 10 hours, about 5 hour to about 9 hours, about 5 hour to about 8 hours, about 5 hour to about 7 hours, about 5 hour to about 6 hours, about 6 hour to about 24 hours, about 6 hour to about 23 hours, about 6 hour to about 22 hours, about 6 hour to about 21 hours, about 6 hour to about 20 hours, about 6 hour to about 19 hours, about 6 hour to about 18 hours, about 6 hour to about 17 hours, about 6 hour to about 16 hours, about 6 hour to about 15 hours, about 6 hour to about 14 hours, about 6 hour to about 13 hours, about 6 hour to about 12 hours, about 6 hour to about 11 hours, about 6 hour to about 10 hours, about 6 hour to about 9 hours, about 6 hour to about 8 hours, about 6 hour to about 7 hours, about 7 hour to about 24 hours, about 7 hour to about 23 hours, about 7 hour to about 22 hours, about 7 hour to about 21 hours, about 7 hour to about 20 hours, about 7 hour to about 19 hours, about 7 hour to about 18 hours, about 7 hour to about 17 hours, about 7 hour to about 16 hours, about 7 hour to about 15 hours, about 7 hour to about 14 hours, about 7 hour to about 13 hours, about 7 hour to about 12 hours, about 7 hour to about 11 hours, about 7 hour to about 10 hours, about 7 hour to about 9 hours, about 7 hour to about 8 hours, about 8 hour to about 24 hours, about 8 hour to about 23 hours, about 8 hour to about 22 hours, about 8 hour to about 21 hours, about 8 hour to about 20 hours, about 8 hour to about 19 hours, about 8 hour to about 18 hours, about 8 hour to about 17 hours, about 8 hour to about 16 hours, about 8 hour to about 15 hours, about 8 hour to about 14 hours, about 8 hour to about 13 hours, about 8 hour to about 12 hours, about 8 hour to about 11 hours, about 8 hour to about 10 hours, about 8 hour to about 9 hours, about 9 hour to about 24 hours, about 9 hour to about 23 hours, about 9 hour to about 22 hours, about 9 hour to about 21 hours, about 9 hour to about 20 hours, about 9 hour to about 19 hours, about 9 hour to about 18 hours, about 9 hour to about 17 hours, about 9 hour to about 16 hours, about 9 hour to about 15 hours, about 9 hour to about 14 hours, about 9 hour to about 13 hours, about 9 hour to about 12 hours, about 9 hour to about 11 hours, about 9 hour to about 10 hours, about 10 hour to about 24 hours, about 10 hour to about 23 hours, about 10 hour to about 22 hours, about 10 hour to about 21 hours, about 10 hour to about 20 hours, about 10 hour to about 19 hours, about 10 hour to about 18 hours, about 10 hour to about 17 hours, about 10 hour to about 16 hours, about 10 hour to about 15 hours, about 10 hour to about 14 hours, about 10 hour to about 13 hours, about 10 hour to about 12 hours, about 10 hour to about 11 hours, about 11 hour to about 24 hours, about 11 hour to about 23 hours, about 11 hour to about 22 hours, about 11 hour to about 21 hours, about 11 hour to about 20 hours, about 11 hour to about 19 hours, about 11 hour to about 18 hours, about 11 hour to about 17 hours, about 11 hour to about 16 hours, about 11 hour to about 15 hours, about 11 hour to about 14 hours, about 11 hour to about 13 hours, about 11 hour to about 12 hours, about 12 hour to about 24 hours, about 12 hour to about 23 hours, about 12 hour to about 22 hours, about 12 hour to about 21 hours, about 12 hour to about 20 hours, about 12 hour to about 19 hours, about 12 hour to about 18 hours, about 12 hour to about 17 hours, about 12 hour to about 16 hours, about 12 hour to about 15 hours, about 12 hour to about 14 hours, about 12 hour to about 13 hours, about 13 hour to about 24 hours, about 13 hour to about 23 hours, about 13 hour to about 22 hours, about 13 hour to about 21 hours, about 13 hour to about 20 hours, about 13 hour to about 19 hours, about 13 hour to about 18 hours, about 13 hour to about 17 hours, about 13 hour to about 16 hours, about 13 hour to about 15 hours, about 13 hour to about 14 hours, about 14 hour to about 24 hours, about 14 hour to about 23 hours, about 14 hour to about 22 hours, about 14 hour to about 21 hours, about 14 hour to about 20 hours, about 14 hour to about 19 hours, about 14 hour to about 18 hours, about 14 hour to about 17 hours, about 14 hour to about 16 hours, about 14 hour to about 15 hours, about 15 hour to about 24 hours, about 15 hour to about 23 hours, about 15 hour to about 22 hours, about 15 hour to about 21 hours, about 15 hour to about 20 hours, about 15 hour to about 19 hours, about 15 hour to about 18 hours, about 15 hour to about 17 hours, about 15 hour to about 16 hours, about 16 hour to about 24 hours, about 16 hour to about 23 hours, about 16 hour to about 22 hours, about 16 hour to about 21 hours, about 16 hour to about 20 hours, about 16 hour to about 19 hours, about 16 hour to about 18 hours, about 16 hour to about 17 hours, about 17 hour to about 24 hours, about 17 hour to about 23 hours, about 17 hour to about 22 hours, about 17 hour to about 21 hours, about 17 hour to about 20 hours, about 17 hour to about 19 hours, about 17 hour to about 18 hours, about 18 hour to about 24 hours, about 18 hour to about 23 hours, about 18 hour to about 22 hours, about 18 hour to about 21 hours, about 18 hour to about 20 hours, about 18 hour to about 19 hours, about 19 hour to about 24 hours, about 19 hour to about 23 hours, about 19 hour to about 22 hours, about 19 hour to about 21 hours, about 19 hour to about 20 hours, about 20 hour to about 24 hours, about 20 hour to about 23 hours, about 20 hour to about 22 hours, about 20 hour to about 21 hours, about 22 hour to about 24 hours, about 22 hour to about 23 hours, or about 23 hour to about 24 hours)

In certain embodiments, the image sensor can be a CMOS or CCD. In a particular embodiment, the image sensor is part of a mobile device with a data connection with the portable handheld imaging device of the present disclosure. For example, the data connection can be Bluetooth, WiFi, ZigBee™, a data port, a data plug, a wire, a USB wire, or any other data connection known to one skilled in the art. In a particular embodiment, the camera is configured to acquire raw laser speckle images. In certain embodiments, the camera takes an RGB image and/or a digital picture. In certain embodiments, the image sensor or camera in combination with light sources and imaging algorithms can be used to acquire a 3D surface image, a virtual image, and/or partial depth measure. In another embodiment, the at least one acquisition device further includes a camera.

The mobile device can be any compact computing device, e.g. a cellular phone (e.g., a smart phone), a tablet, a laptop, a computer (e.g., laptop or desktop), portable apparatus with a display screen, or any other mobile and portable device known to one skilled in the art. The mobile device can include a screen for viewing information and/or an input device. The display and/or input device can be a touchscreen. The mobile device has an operating system that can run various types of application software (e.g., apps) and be equipped with Wi-Fi, Bluetooth, ZigBee™, near field communication (NFC), any data port know to one skilled in the art (e.g., USB), a global position system (GPS), and/or a camera (e.g., CMOS or CCD camera). The mobile device has a power source (e.g., battery) to provide power for the operation of the mobile device, and optionally, peripheries attached thereto (e.g., the handheld device or system). In an embodiment, the mobile device displays a GUI for the device or system. The GUI can be configured to allow a user to select the imaging modalities/analyses to be performed on the region of interest. The GUI can be configured to allow the user to input clinical information regarding the subject, access the subject's medical records, and/or link the imaging to the subject's medical records. The GUI can be configured to present an overlay screen. The overlay screen could include a listing, a graphical representation, or small version of each of the images available for overlay (e.g., raw image(s) and processed image(s)). As such, the user can then select which images are to be overlaid. Alternatively, the user may view the images individually. It should be noted that any mobile device known to one skilled in the art can be utilized in the implementation of the invention of the present disclosure.

In some embodiments, the portable handheld device or system further comprises a screen for display of information and/or an input device. The screen can display a GUI as described above. In this embodiment, the handheld device or system can function independently of or in concert with a mobile device. A handheld device or system of the present disclosure with a screen and input device allows a user to utilize the handheld device or system without the use of a mobile device. In a particular embodiment, the display and/or input device is a touchscreen. In another embodiment, the handheld device or system is operated with a remotely located mobile device. A user may operate the handheld device or system, and the acquired and/or processed data is accessed from a remotely located mobile device through, for example, Bluetooth, Wi-Fi, ZigBee™, or any other appropriate wireless communication methodology.

In another embodiment, the portable handheld device or system can wirelessly communicate with a remotely located computer screen and/or system for display of information, and/or an input device. The remote screen can display a GUI as described above. In an embodiment, the handheld device or system can function independently of or in concert with any device(s) and/or system(s) that have a display screen and an ability to receive data, which is pertinent to advances made in remote care and medical robotics.

According to another aspect of the disclosure a system for performing imaging of biological tissue is provided. In an embodiment, the system comprises: a mobile device with a camera, a source assembly, at least one acquisition device for capturing images of a region of interest of a subject, and a control module. The camera of the mobile device can be configured to capture images of the region of interest, e.g. raw laser speckle images and/or photographic images. The source assembly can include at least one light source. The at least one acquisition device for capturing images of the region of interest can include an IR sensor, RGB sensor, depth sensor, thermographic sensor, a camera, and/or a hyperspectral image sensor. In a particular embodiment of the system, the control module is configured to receive and process laser speckle images from the camera of the mobile device and/or the system, and the hyperspectral image sensor data from the hyperspectral snapshot imager. In an embodiment, the handheld device is configured to communicate wirelessly with the control module comprising non-transitory computer readable medium configured to execute instructions for analyzing data the at least one acquisition device including the camera, to evaluate tissue pathology, tissue healing, and/or regeneration (e.g., a wound at different stages of healing).

The device and system of the disclosure can perform a plurality of modes (i.e., processes or analyses). In an embodiment, a first mode performs a texture and/or color/pigmentation analysis. A picture/image (e.g., a digital picture or RBG image) of the area of interest is taken with the camera of the handheld device, system, or mobile device, as described above, or the hyperspectral image sensor. In an embodiment, the camera and hyperspectral image sensor detects wavelengths in the visible and near infrared light. The picture is analyzed via a texture-color imaging algorithm within the handheld device, system, and/or mobile device, which can differentiate between natural pigmentation, an eschar (a slough or piece of dead tissue that is case off from the surface of the skin—contains necrotic tissue), a scab, and diseased tissue. For example, an area of interest is illuminated with at least one LED or an LED array emitting white light (e.g., about 380 nm to about 730 nm or about 400 nm to about 700 nm) and an image is taken as described above.

In another embodiment, a second mode performs a tissue viability analysis. Red blood cells have strong adsorption of green wavelengths (e.g., about 495 nm to about 570 nm, such as about 510 nm) and weak adsorption of red wavelengths (e.g. 620 nm to about 750 nm, such as about 650 nm). In certain embodiments, polarized white light from the at least one light source (such as at least one LED or an LED array) illuminates a region of interest with a camera and/or the hyperspectral imager acquires at least one image in which the adsorption of green and red wavelengths are analyzed. In a particular embodiment, a polarizing filter is placed in the light path, such that region of interest is illuminated with polarized white light. In an embodiment, the light is polarized, e.g., with a polarizing film and/or lens. Polarized light allows for a better distinction between normal tissue pigmentation (melanin) and diseased tissue (e.g., melanoma), and in combination with appropriate optical elements and a contrast based processing algorithm, tissue viability can be analyzed by this method. The device or system can include a second polarizing filter configured to filter out light that interacts with the surface of the skin and allows scattered light to pass through to be detected by a camera and/or hyperspectral imager. In another embodiment, the region of interest is illuminated with polarized light and variations in contrast can be analyzed, as a byproduct of light waves reflecting from the area of interest deflect onto the imager/camera filters. Light waves that are perpendicular to the filters aligned at the camera/image sensor are canceled (i.e., stopped by the filter(s) and the scattered light passes through the filter(s) to the imager, as described above. The scattered light allows for differentiation between normal skin pigmentation and diseased tissues. In an embodiment, the image is acquired, processed, and analyzed as described in Jacques et al. Imaging superficial tissues with polarized light, Lasers Surg. Med. 26:119-129. In a particular embodiment, the area of interest is illuminated with polarized light in the visible range, and the relative absorption of red and green wavelengths are compared to determine tissue perfusion (i.e., red blood cell concentration in the imaged tissue). The red blood cell concentration is utilized to make a tissue viability determination. For example, tissue that has no perfusion is not viable, while relatively low perfusion (i.e., less perfusion than normal tissue) would indicate that there may be a tissue viability issue. In an embodiment, the processing of tissue viability data is performed as described in Zhang et al. (Multimodal imaging of cutaneous wound tissue. Journal of Biomedical Optics. 20(1): 016016 (January 2015)).

According to another embodiment, a third mode performs a tissue oxygenation analysis and/or results in a tissue oxygenation map of the area of interest. A hypercube is taken of the area of interest with the hyperspectral image sensor, as described above. The light can be provided by at least one LED or an LED array. The tissue oxygenation analysis is performed by the control module of either the handheld device or the system, or the processor of the mobile device. In a particular embodiment, the hyperspectral image data is processed with the FGPA, the GPU, or the FGPA and the GPU. The tissue oxygenation analysis includes algorithms (tissue oxygenation analysis algorithms) that decompile the hypercube, interpret the decompiled hypercube data, and reconstruct a spectral map. The spectral map can highlight bands that have been used to evaluate tissue health and/or disease, and as described above, presented to the user. For example, the hyperspectral imager can detect the emission spectra of diabetic ulcers excited by light in a range of about 570 nm to about 630 nm (e.g. about 600 nm). By way of another example, the hyperspectral imager can detect the emission spectra of a chromophore. For example, the emission spectra of oxyhemoglobin/deoxyhemoglobin excited by light in a range of about 770 nm to about 815 nm (e.g., about 780 to about 805 nm) can be detected. Furthermore, the hyperspectral imager can detect the emission spectra of tissue malignancies excited by light at a wavelength in a range of about 840 nm to about 2,500 nm (e.g., about 850 nm to about 1,200 nm). Accordingly, in an embodiment, the region of interest is excited at a plurality of wavelengths via at least one LED and/or an LED array emitting visible and/or near infrared light (e.g., about 400 nm to about 2,500 nm).

Again, the control module of either the handheld device or the system, or the processor of the mobile device acquires, extracts, decompiles (unmixes), and processes the hypercube data. In an embodiment, the hyperspectral image data may contain 5×5 pixels that include data for the various spectral wavelengths. Decompiling or unmixing can be accomplished by defining skewers and performing a dot product across all spectral values of each pixel. A skewer is a normalized vector that defines a spectral footprint of a material for specified spectral bands. The device or system of the present disclosure performs the processing/analysis in real-time or close to real-time. In an embodiment, the processing/analysis of the hyperspectral image data occurs in about 30 seconds or less. In a particular embodiment, the processing/analysis of the hyperspectral imaging data occurs in about 1 second to about 30 seconds, about 1 second to about 25 seconds, about 1 second to about 20 seconds, about 1 second to about 15 seconds, about 1 second to about 10 seconds, about 1 second to about 5 seconds, about 5 seconds to about 30 seconds, about 5 seconds to about 25 seconds, about 5 seconds to about 20 seconds, about 5 seconds to about 15 seconds, about 5 seconds to about 10 seconds, about 10 seconds to about 30 seconds, about 10 seconds to about 25 seconds, about 10 seconds to about 20 seconds, about 10 seconds to about 15 seconds, about 15 seconds to about 30 seconds, about 15 seconds to about 25 seconds, about 15 seconds to about 20 seconds, about 20 seconds to about 30 seconds, about 20 seconds to about 25 seconds, or about 25 seconds to about 30 seconds. A two-dimensional map conveying the health of the tissue in the region of interest is constructed. The two-dimensional map can outline the health of the tissue throughout the lesion, e.g. the boarders and interior portions of the lesions. In a particular embodiment, the hyperspectral data is acquired, processed, and/or analyzed as described in Chen et al. (Development of a Thermal and Hyperspectral Imaging System for Wound Characterization and Metabolic Correlation. Johns Hopkins APL Technical Digest, 2005. 26(1)). In another embodiment, the hyperspectral data is processed as described in Gonzalez et al. (Use of FPGA or GPU-based architectures for remotely sensed hyperspectral image processing. Integration, the VLSI journal 46 (2013) 89-103). Although specific analyses are described herein, it should be noted that the hyperspectral image sensor and at least one light source can be utilized to perform a hyperspectral analysis to detect and assess other tissue pathologies, for example, as described in Lu et al. (Medical hyperspectral imaging: a review. Journal of Biomedical Optics 19(1), 010901 (January 2014)). For example, the following tissue pathologies may be assessed: burn wounds at about 400 nm to about 1,100 nm, a diabetic foot at about 500 nm to about 600 nm and/or about 400 nm to about 720 nm, or melanoma at about 365 nm to about 800 nm. The hyperspectral image sensor can also be utilized to determine the molecular context of the tissue. For example, the content of water, lipids, and/or melanin at different tissue depths can be determined. In an embodiment, the hyperspectral image data is acquired, processed and/or analyzed as described in Zhang et al. (Multimodal imaging of cutaneous wound tissue. Journal of Biomedical Optics 20(1), 0106016 (January 2015)) to determine tissue oxygenation.

In another embodiment, a fourth mode performs a 3D image reconstruction of the surface of the region of interest, e.g., a wound. The device or system can illuminate the region of interest with visible light, as described above, and the region of interest is imaged, e.g, simultaneously, with the hyperspectral imager, light sources, and the camera of the device, system or mobile device. The device or system processes the camera and hyperspectral image sensor data to produce a virtual 3D reconstruction of the area of interest (e.g., a wound). In an embodiment, the surface volume of a wound in the region of interest is determined, which can be used as a marker of tissue regeneration, and/or tissue healing. The surface volume of a wound can be used to log the changing dimensions of the tissue area. For example, negative surface volume can be correlated to the progress of wound healing. For example, a smaller negative surface volume compared to a previous scan could indicate tissue regeneration and tissue healing. Similarly, a larger negative surface volume compared to a previous scan could indicate progression of tissue pathology or the presence of a disease. Furthermore, relative surface volume can be correlated to a degree of tissue pathology and therefore, an estimate of time required for the tissue pathology to heal. 3D reconstructions can be surface and sub-surface, which relates to the particular light source and wavelengths and image sensors. In a particular embodiment, the light based 3D surface imaging allows for a 3D surface reconstruction of contours and dimensions of tissue pathology, tissue healing, and/or regeneration. In a particular embodiment, the use of optical components, light sources, sensors, and camera(s) and computer visions systems as they relate to 3D surface image processing and analysis is described in Bills et al. (Pilot study to evaluate a novel three-dimensional wound measurement device, International Wound Journal, 2015).

In another embodiment, a fifth mode performs a microvascular analysis. A laser speckle image is taken of the region of interest with the camera (e.g., a CMOS camera) located on the handheld device, the system, or the mobile device, or the hyperspectral image sensor). The microvascular analysis is performed by the control module of either the handheld device or system (e.g., the FPGA, the GPU, or both), or the processor of the mobile device. The microvascular analysis includes at least one microvascular analysis algorithm, e.g. laser speckle imaging algorithms and/or particle velocimetry imaging algorithms. In an embodiment, the camera or hyperspectral image sensor (e.g., a snapshot hyperspectral imager) takes a video of backscattered light. The video can include a speckle pattern that is processed by the algorithms to produce a three-dimensional reconstruction of the microvasculature. In an embodiment, the device comprises an image projector, and the three-dimensional reconstruction (or an imaged derived therefrom, e.g., a two dimensional representation of the reconstruction) is projected on the region of interest. 3D reconstructions can be surface and sub-surface, which relates to the particular light source and wavelengths and image sensors. In a particular embodiment, the hyperspectral imager and additional camera(s) will be leveraged with the appropriate imaging algorithms to reconstruct microvasculature that is within and slightly below the skin surface and tissue borders, contours, and dimensions that are exposed at the surface level to the outside world. In a particular embodiment, the use of optical elements, coherent light sources, hyperspectral imager and/or additional camera sensors data is processed and analyzed to analyze microvascular architecture and health as described in Rege et al. Rege (In vivo laser speckle imaging reveals microvascular remodeling and hemodynamic changes during wound healing angiogenesis. Angiogensis, 2012).

For example, light can be provided to the region of interest at a wavelength in a range of about 670 nm to about 695 nm (e.g., 688 nm) and/or a range of about 780 nm to about 805 nm (e.g., about 795 nm). Each wavelength range can be provided by at least one laser (e.g., a laser diode, a VCSEL or a VSCEL array), or at least one LED (e.g., an LED array). VSCELs provide a substantially coherent light (i.e., a single frequency) that is more uniform and coherent than laser diodes. VCSELs also have a reduced speckle pattern, thereby providing more dependable laser speckle data in variable lighting environments. Hemoglobin absorbs coherent light at a wavelength of about 780 nm to about 805 nm (e.g., about 795 nm). As such, one can use laser speckle to track the movement of hemoglobin, e.g. through microvessels (such as arterioles, capillaries, metarterioles, venules, etc.).

In an embodiment, a diffuser (e.g., microdiffuser) reflects the light emitted from the VCSEL(s) or VCSEL array to provide a uniform coherent light to the region of interest. The light provided by the VCSEL(s) or the VCSEL array(s) can reflect off of the diffuser to the region of interest. The VCSEL or VSCEL array and the diffuser provide a more uniform light than is provided by standard laser diodes. The angle of incidence of the light (e.g., about 670 nm to about 695 nm) on the diffuser will determine the width of the light on the region of interest, as well as the distance required between the device/system and the region of interest. For example, in a particular embodiment, the angle of incidence of the VCSEL light is such that the device/system is located in a range of 0 cm to about 20 cm from the region of interest. In a certain embodiment, the about 670 nm to about 695 nm light and the about 780 nm to about 795 nm light are positioned to have areas of reflection (e.g., the region of interest) that are substantially the same.

The laser speckle image data can be processed to determine the microvasculature of the region of interest by any means one skilled in the art would appreciate. In a particular embodiment, the microvascular analysis is performed and processed as described in Yang et al. (Real-time blood visualization using the graphics processing unit. Journal of Biomedical Optics 16(1), 016009 (January 2011)) or Juric and Zalik (An innovative approach to near-infrared spectroscopy using a standard mobile device and its clinical application in the real-time visualization of peripheral veins. BMC Medical Informatics and Decision Making 2014, 14: 100).

In an embodiment, the processing/analysis of the hyperspectral data, the laser speckle data, and the 3D surface reconstruction data occurs in about 30 seconds or less. In a particular embodiment, the processing/analysis of the hyperspectral data, the laser speckle data, and the 3D surface reconstruction data occurs in about 1 second to about 30 seconds, about 1 second to about 25 seconds, about 1 second to about 20 seconds, about 1 second to about 15 seconds, about 1 second to about 10 seconds, about 1 second to about 5 seconds, about 5 seconds to about 30 seconds, about 5 seconds to about 25 seconds, about 5 seconds to about 20 seconds, about 5 seconds to about 15 seconds, about 5 seconds to about 10 seconds, about 10 seconds to about 30 seconds, about 10 seconds to about 25 seconds, about 10 seconds to about 20 seconds, about 10 seconds to about 15 seconds, about 15 seconds to about 30 seconds, about 15 seconds to about 25 seconds, about 15 seconds to about 20 seconds, about 20 seconds to about 30 seconds, about 20 seconds to about 25 seconds, or about 25 seconds to about 30 seconds.

As discussed above, the heterogeneous computational board allows for additional sensors to be added. For example, an inflammatory reaction analysis may be performed by connecting a thermographic sensor to the device. For example, a thermographic sensor could be part of the system or connected to the handheld device or system through the communication/data port or plug. As such, the thermographic sensor images the region of interest. The heat signature of the region of interest is processed and analyzed by the FPGA to determine if an inflammatory response is present in the tissue pathology. For example, if the tissue pathology is warmer than the surrounding tissue, an inflammatory response is present. Furthermore, the larger the area of the heat signature relative to the tissue pathology and/or the greater the differential between the heat signature and the surround tissue, the more substantial the inflammatory response. The processing can be performed by any means known in the art. For example, the thermographic sensor data can be performed and processed as described in Zhang et al. (Multimodal imaging of cutaneous wound tissue. Journal of Biomedical Optics 20(1), 0106016 (January 105)) to determine whether there is, and to what extent there is, an inflammatory response present in at the site of tissue pathology.

In an embodiment, an image (e.g., a picture or a basic digital image) of the region of interest is taken. This image can be a digital image taken from the camera (e.g., a CCD or CMOS camera) of the device, system or mobile device, or the hyperspectral image sensor. In an embodiment, the control module of the device or system, or the processor of the mobile device registers each of the processed images (i.e. the analyzed data images) and/or raw images as described throughout the present disclosure. The control module can then overlay as many of the raw data, analyzed data images, and/or the captured image of the region of interest as the user would like through the GUI as described above. Registering of images can be performed by any means known in the art. For example, in an embodiment, image registration is performed as described in Zhang et al. (Multimodal imaging of cutaneous wound tissue 10(1), 016016 (January 2015).

In some embodiments, a large image file (e.g., the three dimensional representation of the analyzed hyperspectral data) is converted to a smaller image file (e.g., a compressed image file format and/or the same image file format with less detail), which is overlaid with other images or individually presented on the screen of the handheld device, the system and/or the mobile device. Each of the images produced through the above discussed analyses can be presented overlaid with a subset or all of the other images (analyzed or raw) or individually on the screen of the device, system or mobile device.

In another embodiment of the present disclosure, the mobile device controls the at least one light source. In other embodiments, the mobile device controls the at least one acquisition device (e.g., a plurality of acquisition devices), which can include a camera on handheld device or system, and the camera of the mobile device. In another embodiment, the control module controls the source assembly and/or the plurality of acquisition devices, which can include a camera of the mobile device, the device, and/or system. The mobile device and control module may control the at least one light source and/or the at least one acquisition device via microcontrollers as described above.

According to another aspect of the disclosure is a method of evaluating tissue pathology is provided. The method comprises providing a device or system as described herein with or without a mobile device. For example, the method may comprise: providing a mobile device having a camera for acquiring raw laser speckle images, a source assembly including at least one light source, at least one acquisition device including a hyperspectral image sensor, and a control module. The method further comprises acquiring the raw laser speckle image, and hyperspectral image data of a tissue region of interest of a subject in need thereof, and analyzing the tissue region of interest, and optionally treating or diagnosing the subject having a tissue pathology (e.g., a wound).

In an embodiment, analyzing comprises processing the raw laser speckle image/data, thermal image/data, and/or a digital picture to provide a microvascular analysis (e.g., a three dimensional reconstruction of the microvascular) of the region of interest, the degree of inflammation at the region of interest, and/or a texture color analyzed image. The texture color analyzed image differentiates between natural pigmentation, eschars, scabs, and diseased tissue. In another embodiment, the hyperspectral image/data is processed to provide a spectral map of, for example, ulcers (e.g., diabetic ulcers, pressure ulcers, vascular ulcers, traumatic wounds, post-surgical wounds, and non-healing wounds), oxyhemoblogin/deoxyhemoglobin, and malignancies.

In an embodiment, the control module of the device or system is configured to send the processed image data to a processing center. The processing center can be a cloud based system, a server, or a computer. The processing center can perform pattern recognition on the individual images or a group of images to make a diagnosis recommendation or a treatment recommendation. In an embodiment, the processing center is remotely located, for example, in a different room, building, geographical location, or country. As such, the device and system of the present disclosure provide telemedicine capabilities.

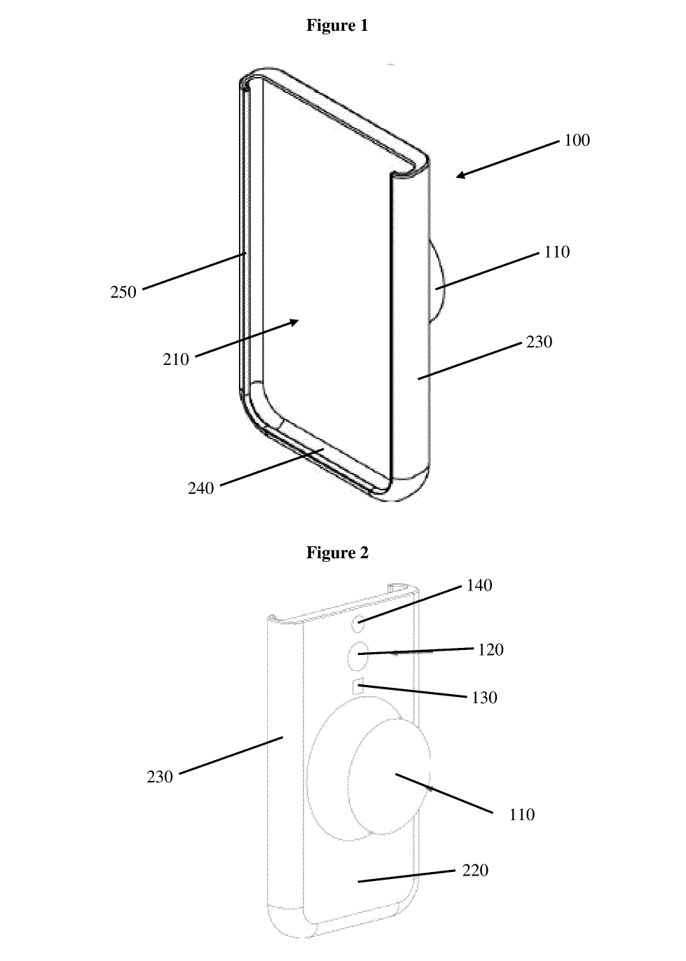

FIG. 1 illustrates a handheld device 100 in accordance with an embodiment of the present disclosure. The handheld device 100 includes a cradle 210 which comprises a base 220, side walls 230, a bottom wall 240, and retention pieces 250.

FIG. 2 illustrates a handheld device 100 in accordance with an embodiment of the present disclosure. The handheld device 100 includes at least one acquisition device 110 (e.g., a hyperspectral image sensor), at least one light source 120, 130, and a camera 140. One skilled in the art would appreciate that the order and location of the at least one light source and the at least one acquisition device (e.g., a camera) can be modified.

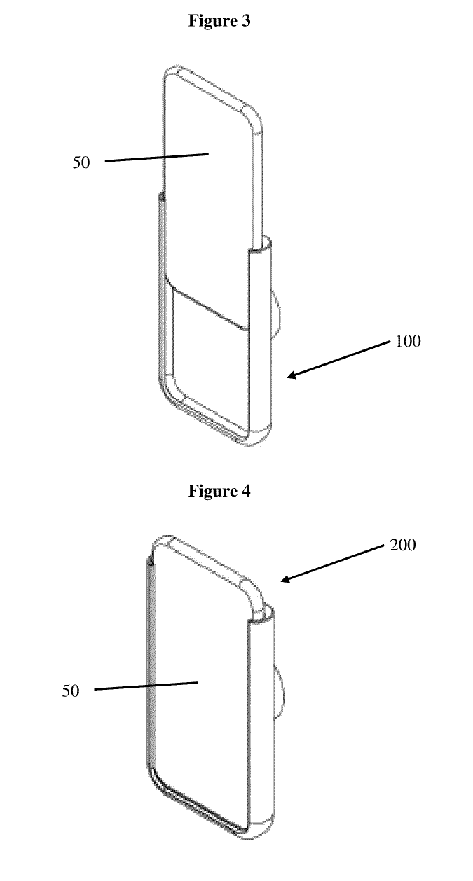

FIG. 3 is an illustration of a handheld device 100 in accordance with an embodiment of the present disclosure and a mobile device 50 being inserted into the cradle 210 of the device.

FIG. 4 is an illustration of a handheld device 100 with a mobile device 50 located in a cradle 210 of the handheld device 100 or a system 200 in accordance with an embodiment of the present disclosure.

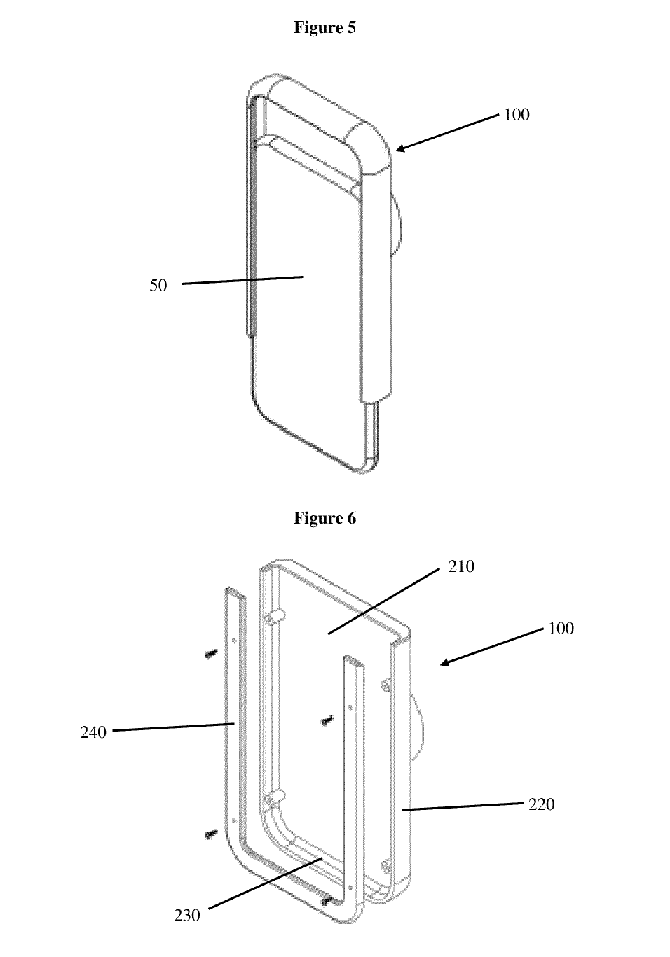

FIG. 5 illustrates a handheld device 100 in accordance with an embodiment of the present disclosure and a mobile device 50 being inserted into the cradle 210 of the device.

FIG. 6 illustrates a handheld device 100 in accordance with an embodiment of the present disclosure wherein the retention pieces 250 are attached by, e.g., screws.

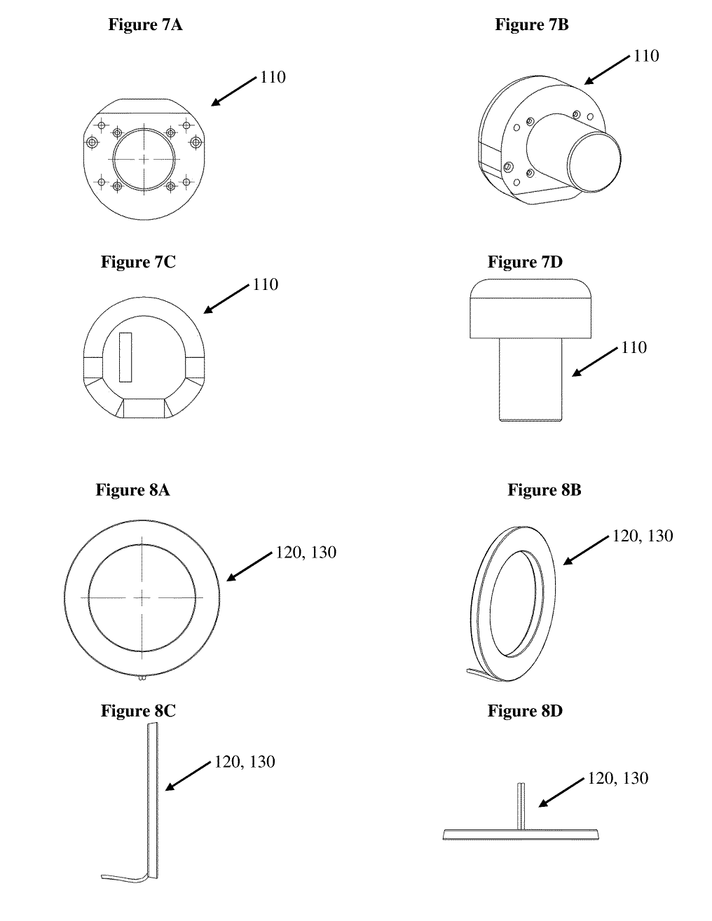

FIGS. 7A, 7B, 7C, and 7D illustrate several perspectives of a hyperspectral imager 110 used in a device of the present invention.

FIGS. 8A, 8B, 8C, and 8D illustrates a light source 120, 130 used in a device of the present invention. In particular, FIG. 8 illustrates several perspectives of a LED ring 120, 130, which can be an LED array.

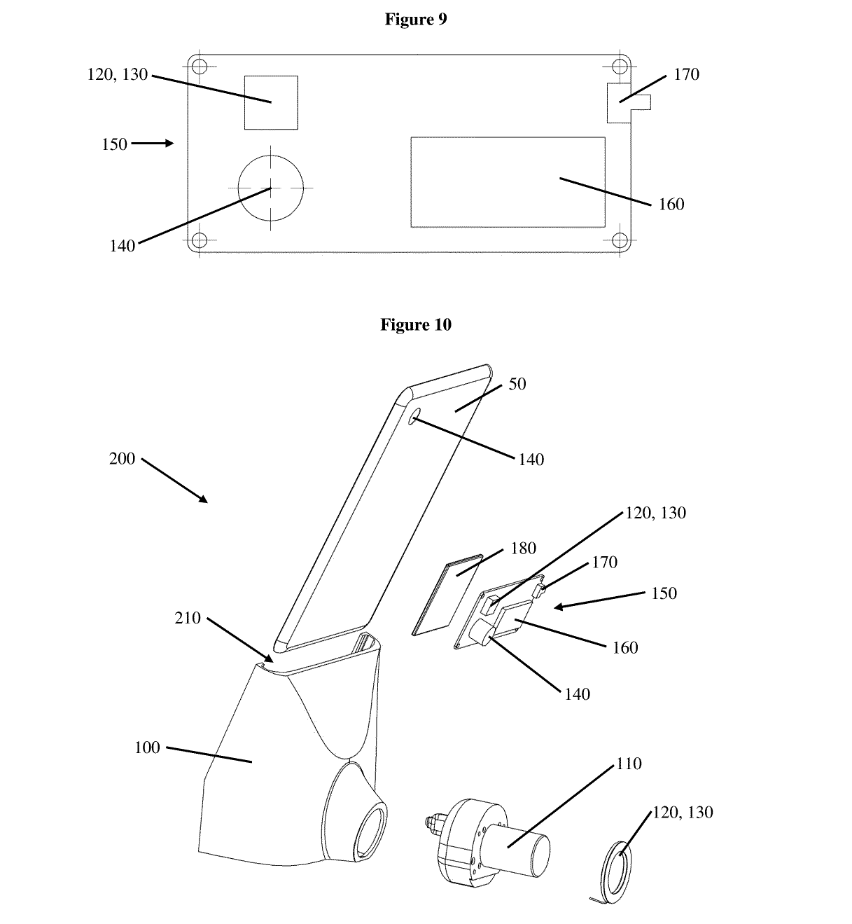

FIG. 9 illustrates a heterogeneous computational board 150 with a FPGA 160. The heterogeneous computation board 150 may also include an on/off switch 170, a light source 120, 130 (e.g., an infrared light source), and a camera 140 (e.g., an infrared camera).

FIG. 10 illustrates a system 200 in accordance with an embodiment of the invention. The system includes a portable device 100 and a mobile device 50. The particular device is shown with a heterogeneous computational board 150 as shown in FIG. 9, a battery 180, a hyperspectral imager 110, and an LED ring 120, 130. The portable device of FIG. 10 includes a hyperspectral imager 110 and a camera 140 (e.g., a visible, and IR imager). The portable device of FIG. 10 includes two light sources—a laser 120,130 on the heterogeneous computation board 150 and a LED ring 120,130. In an embodiment, laser comprises a VSCEL array or discrete VSCELs (e.g., a first VSCEL and a second VSCEL) that emit light in a range of about 630 nm to about 805 nm (e.g. about 688 nm) and in a range of about 780 nm to about 795 nm (e.g., about 795 nm). In another embodiment, the LED ring is a multi-LED array of 4 LEDs to 30 LEDS (e.g., 4, 5, 6, 7, 8, 9, 10, 11, 12, 13, 14, 15, 16, 17, 18, 19, 20, 21, 22, 23, 24, 25, 26, 27, 28, 29, or 30 LEDs). In a particular embodiment, an LED array (or discrete LEDs) emit light in a range of about 570 nm to about 630 nm (e.g., 600 nm), a range of about 630 nm to about 695 nm (e.g., about 688 nm), a range of about 770 nm to about 815 nm, and a range of about 850 nm to about 2,500 nm. As discussed above, in another embodiment, the device 50 does not include a camera 140. The mobile device 50 is depicted with a camera 140. In a particular embodiment, a fisheye camera 140 of a mobile device 50 takes a RGB image of the region of interest, an IR camera 140 takes a laser speckle image of the region of interest, and a hyperspectral camera 110 takes a hyperspectral image of the region of interest. In a certain embodiment, an IR laser 120, 130 illuminates the region of interest for the laser speckle image. In another embodiment, an LED array 120, 130 is illuminates the region of interest for the hyperspectral image.

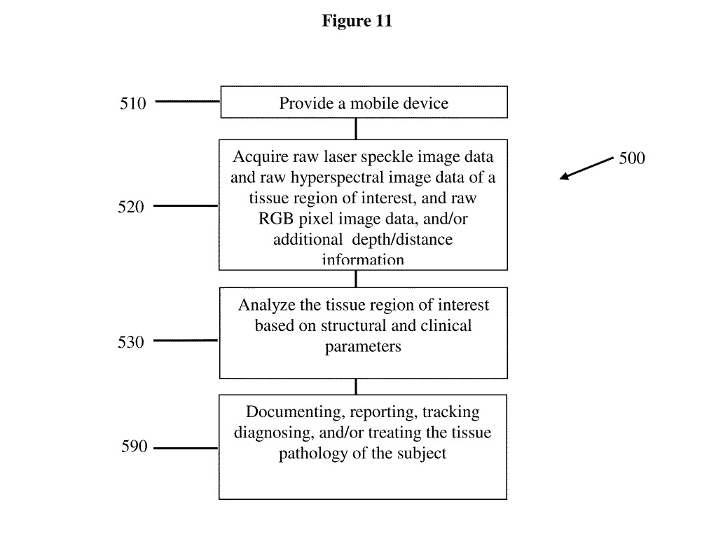

FIG. 11 is a flow diagram of an embodiment of a method 1000. The method comprising: providing a mobile device 510; acquiring raw laser speckle image data and raw hyperspectral image data and raw RGB pixel image data and/or additional depth sensing information of the tissue region of interest 520; analyzing the tissue region of interest 530; and optionally, documenting, reporting, tracking, diagnosing and/or treating the tissue pathology of the subject. It should be noted that the diagnosis and treatment decision may be made remotely—for example, telemedicine.

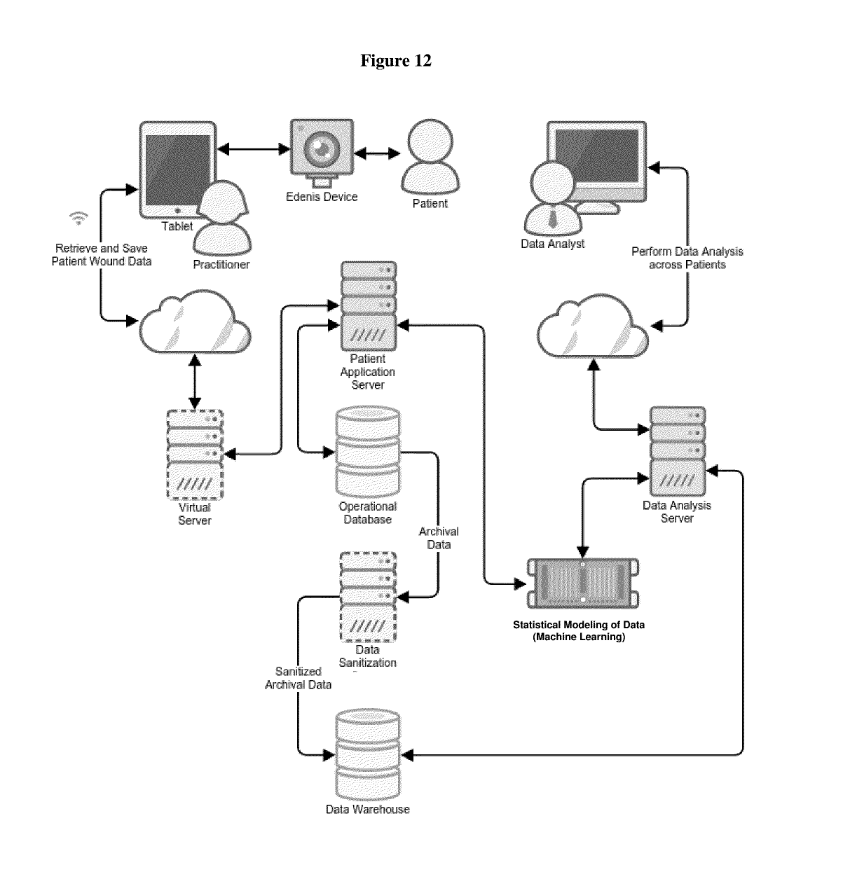

FIG. 12 is a diagram of the As discussed above, a device that contains one or more sensors and transmits sensors data either in its original state or after processing to an adequately equipped mobile device (e.g., a smartphone, tablet, or a 2-in-1 computer) via a network connection (wifi, bluetooth, or a physical connection, such as data plug and port). In an embodiment, software on the mobile device performs at least one of: controls the sensor device; transmits sensor data to a cloud-based service for persistence; retrieves previously persisted data (either by the same or a different device) from the cloud-based service; transfer persists data to the mobile device itself; processes the device data for user consumption; and displays and affords user-interactivity with the sensor data or data derived from it.

In an embodiment, a cloud-based software service can be provided that performs at least one of: enforces data access control via authentication and authorization to (store data from the mobile device to a persistent storage medium and retrieves data from persistent storage and return it to a mobile device upon request); interfaces with a machine learning program to analyze the sensor data against a data model for the purpose of providing health care providers with actionable clinical data, diagnostic support and recommendations (optionally analyzing the sensor data with deep learning as a sub-category of machine learning) to analyze aggregated image data and to investigate visual and tissue patterns; analyzes multiple sets of sensor data per patient to provide time-based progression/regression information; correlates sensor data with clinical meta data (which may include demographic, laboratory, procedural, administrative); transfers data out of the production database to a data warehouse (optionally sanitizing the data of personally identifying information); and performs trend analyses on multiple sets of sensor data and correlated clinical data to determine important co-factors of progress/regression.

The embodiment of tissue disease and tissue healing and tissue regeneration monitoring algorithms involves the following steps:

Modes 1 and 2. In one embodiment of the algorithm set performs digital images, RGB texture/color analysis:

1. Acquire digital image.

2. Align coordinates by way of software to determine an area of interest, align with coordinates established with light elements.

3. Conduct RGB and texture/analysis within mobile app, which can include border dimensions, color variations, erythema (redness) scaling, pigmentation variations, and/or light reflectance differences.

4. Overlay this image with additional device image.

Mode 3. In one embodiment of the algorithm set performs multi-spectral and hyperspectral analysis:

1. Acquire the data and interpret: Read raw data pixel by pixel. Spectral unmixing. Apply skewer to normalize spectral footprint. DSP's perform a multiplication of the skewer that pertains to a given pixel at a specific wavelength.

2. Preprocess the HSI data. In an embodiment, remove background radiation by subtracting the calibrated background radiation from each newly acquired image, while accounting for uneven light distribution.

3. Post-process the HSI data. Align coordinates to determine area of interest, align with coordinates established by way of light source region and software area designations.

4. Define and analyze region of interest (ROI). In an embodiment, the solution is to be calculated unless the entire field of view to be analyzed. Leverage software to coordinate analyze with region of interest. Convert all hyperspectral image intensities into units of optical density. Tissue sites include connective tissues, oxygenated tissues, muscle, tumor, and blood.

5. Decompose the spectra for each pixel (or ROI averaged across several pixels). In an embodiment, decompose into several independent components, more preferably, two of which are oxyhemoglobin and deoxyhemoglobin. In some instances, a video based frame rate capture will present sufficient hypercubes to evaluate microvasculature when combined with LSCI.

6. Determine planes for color and/or pseudo-color images. In an embodiment, determine by using characteristic features of the spectra as they relate to tissue sites and characteristics.

7. Remove outliers in the resulting image, defining an outlier as color intensity deviating from a typical range beyond certain number of standard deviations.

8. Compress and package 3D findings/data into 2D image map depicting metabolic states.

9. Transfer the report to the mobile device, the display of the device/system, and/or remote display screen for presentation as a single layer within a multiple layer image presentation.

Modes 4 and 5: In one embodiment of the algorithm set performs laser speckle contrast analysis and 3D surface image analysis

1. Acquire digital image and/or IR sensor image.

2. Evaluate with the respective light sources (lasers and/or laser projector and/or LED).

3. Align coordinates by way of software and optical elements (e.g., laser) to determine area of interest.

4. Conduct laser speckle analysis by way of algorithms, mobile app or device or a combination of both, and optical elements (e.g., lasers) which denotes blood supply, and microvascular architecture 3 dimensionally.

5. Simultaneously evaluate depth (sensing) and further evaluate the border dimensions, surface contour and architecture, and reconstruct the surface architecture three dimensionally.

Synchronize the two 3D images for precision and represent this image as a layer overlay with the other images, and/or as the primary visual aid for the mobile and handheld device users/handlers and/or the clinical user/data analyst analyzing findings at a remote location.

Optional Modes (e.g., Adding peripherals or refining algorithms): For instance, in another embodiment of the algorithm set thermographic image analysis.

Overlay of Distinct Modes. The distinct modes, though represented sequentially, can occur in parallel and/or in any combination of sequences, as discussed above:

1. Display region of interest in resultant image overlay form. For instance, tissue oxygenation maps within tomographic images and represented microvasculature to reinforce anatomy pertaining to area of interest.

2. Data structuring and characterization of the tissue condition, viability, and progression of disease or healing and/or regeneration.

In this present invention, a new multimodal imaging device, system, and methods are presented to address the aforementioned problems, which encompass the areas of imaging technologies, mechanical designs, electrical considerations, board-level configurations, and software. In the present invention, a new device, system, and methods are presented that allow the assessment of tissue structure, pathology, viability and healing and/or regeneration. Tissue disease detection can include the detection of necrosis, poor or lack of blood supply, de-oxygenated tissues, delayed healing/regeneration, scarring, abnormal growth, structural alterations, malignant tissues and cells. In an embodiment, a portable handheld multi-modal compact imaging system utilizing optical imaging sensor technologies serves as a tool for clinical assessment, monitoring, and diagnosis of tissue disease and the monitoring and evaluation of tissue healing and regeneration monitoring, thereby providing advances in management and treatment protocols.

This system provides images for further analysis by the human. Initially it is not driven by artificial intelligence as a caregiver and clinical provider would want to make the final decision as it relates to management and treatment. As more information is gathered, however, as a spectral library is compiled and techniques are refined and such information can be evaluated with deep learning and computer learning to investigate patterns and derive statistical significance between specific patterns and clinical information. The system has the capability to become a diagnostic device as it relates to tissue pathology, tissue healing and tissue regeneration. This invention is a novel, elegant, cost effective and ‘true portable’ (handheld) real-time imaging system. It is clear to one skilled in the art that there are many uses for a portable multi-modal imaging device and system. The device offers the advantages of performing the functions for such uses faster, more economically, and with less equipment and infrastructure/logistics tail than other conventional techniques. Many similar examples can be ascertained by one of ordinary skill in the art from this disclosure for circumstances where medical providers rely on their visual analysis of biological tissue. This system acts like an “augmented reality and virtual reality” system to help humans explore non-organic, organic, biological, physiological, anatomical, and cellular spaces.

As would be understood by those of skill in the art, certain quantities, amounts, and measurements are subject to theoretical and/or practical limitations in precision, which are inherent to some of the instruments and/or methods. Therefore, unless otherwise indicated, it is contemplated that claimed amounts encompass a reasonable amount of variation.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims.

What is claimed is:

1. A handheld device for performing imaging of biological tissue, the device comprising a housing including:
a source assembly having at least one light source;
at least one acquisition device for capturing images of a region of interest of a subject, the at least one acquisition device including a hyperspectral image sensor; and
a control module configured to:
communicate with a mobile device with a camera; and
process raw laser speckle image data from the camera, raw digital picture data from either the camera or the hyperspectral image sensor, and raw hyperspectral image sensor data to evaluate tissue pathology and tissue regeneration of the region of interest.

2. The device of claim 1, the control module is further configured to process 3D surface image data to evaluate tissue pathology and tissue regeneration of the region of interest.

3. The device of claim 1, wherein the at least one light source is at least one of a light emitting diode (LED) and a laser.

4. The device of claim 1, wherein the at least one light source comprises two light sources that emits light at a wavelength in a range of about 400 nm to about 2,500 nm, and about 600 nm to about 2,500 nm.

5. The device of claim 4, wherein the about 400 nm to about 2,500 nm wavelength light is emitted from a LED and the about 600 nm to about 2,500 nm wavelength light is emitted from a laser.

6. The device of claim 5, wherein the laser is a vertical cavity surface emitting laser (VCSEL).

7. The device of claim 5, further comprising a diffuser located within a light path of at least one of the at least one laser.

8. The device of claim 5, further comprising a polarizing film located within a light path of at least one of the at least one LED.

9. The device of claim 1, wherein the hyperspectral image sensor is a snapshot hyperspectral imager that acquires the laser speckle image data and/or a video recording.

10. The device of claim 1, wherein the hyperspectral image sensor is a mosaic hyperspectral imager.

11. The device of claim 1, further comprising at least one of a rechargeable battery, a camera of the device, a display of the device, and an input device.

12. The device of claim 1, wherein the control module includes a heterogeneous computational board with a field-programmable gate array (FPGA), a graphic processing unit (GPU), and a central processing unit (CPU).

12. The device of claim 12, wherein the FPGA is configured to process/analyze the hyperspectral image sensor data and/or the laser speckle image data.

14. The device of claim 12, wherein the GPU is configured to process and/or analyze the hyperspectral image sensor data and/or the laser speckle image data.

15. The device of claim 1, wherein the control module is configured to register a plurality of processed data and a plurality of raw data.

16. The device of claim 15, wherein the control module is configured to overlay at least two of the plurality of processed data and the plurality of raw data, and to transmit the overlay to a screen of the mobile device for display or to a screen of the device for display.

17. The device of claim 1, further comprising microcontrollers configured to control the plurality of acquisition devices and the source assembly during the imaging of the region of interest.

18. A system comprising:
a mobile device having a camera for capturing images of a region of interest of a subject including raw laser speckle images; and
a housing comprising:
a source assembly including at least one light source;
at least one acquisition device for capturing images of the region of interest, the at least one acquisition device includes a hyperspectral image sensor; and
a control module configured to receive and process the raw laser speckle images, the raw thermographic sensor data, and raw hyperspectral image sensor data,
wherein the control module is configured to communicate with the mobile device, and includes a processor comprising non-transitory computer readable medium configured to analyze the raw laser speckle images from the camera, and raw hyperspectral image sensor data to detect or assess tissue pathology.

18. The system of claim 18, wherein the processor is further configured to analyze 3D surface image data to detect or assess tissue pathology.

20. The system of claim 18, wherein the at least one light source comprises a LED that emits light at a wavelength in a range of about 400 nm to about 2,500 nm, and a laser that emits light at a wavelength in a range of about 600 nm to about 2,500 nm.

21. The system of claim 18, wherein the control module includes a heterogeneous computational board with a field-programmable gate array (FPGA), a graphic processing unit (GPU), and a central processing unit (CPU).

22. The system of claim 21, wherein the FPGA is configured to process/analyze the hyperspectral image sensor data and/or the laser speckle image data.

23. The system of claim 21, wherein the GPU is configured to process and/or analyze the hyperspectral image sensor data and/or the laser speckle image data.

24. The system of claim 18, wherein the control module is configured to register a plurality of processed data and a plurality of raw data.

25. The system of claim 18, wherein the control module is configured to overlay at least two of the plurality of processed data and the plurality of raw data, and to transmit the overlay to a screen of the mobile device, a screen of the housing, or a remote device for display.

26. The system of claim 18, wherein the hyperspectral image sensor is a snapshot hyperspectral imager that acquires at least one of the laser speckle image data and a video recording.

27. The system of claim 18, wherein:
the at least one source includes:
an LED array that emits light at a wavelength in a range of about 570 nm to about 630 nm, a range of about 630 nm to about 695 nm, a range of about 770 nm to about 815 nm, and a range of about 850 nm to about 2,500 nm;
a first VSCEL that emits light at a wavelength in a range of about 630 nm to about 805 nm; and
a second VSCEL that emits light at a wavelength in a range of about 780 nm to about 795 nm;
the at least one acquisition device further includes a camera that detects at least one of visible, near infrared, and infrared spectrum;
the hyperspectral image sensor is a snapshot hyperspectral imager;
the control module is a heterogeneous computational board with a FPGA, GPU, and a CPU;
the control module is configured to:
process/analyze the laser speckle and/or the hyperspectral image data with the FPGA, the GPU, or the FPGA and the GPU;
register a plurality of processed image data and a plurality of raw image data;
overlay at least two of the plurality of processed data and the plurality of raw data; and
transmit the overlay to a screen of the mobile device, a screen of the housing, or a remote device for display.

28. A method of evaluating tissue pathology and tissue regeneration, the method comprising:
providing a mobile device having:
a camera for acquiring raw laser speckle images;
a source assembly including at least one light source;
at least one acquisition device including a hyperspectral image sensor, and a control module;
acquiring the raw laser speckle image and hyperspectral image of a tissue region of interest of a subject in need thereof; and
analyzing the tissue region of interest, and optionally treating or diagnosing the subject having tissue pathology.

29. The method of claim 28, wherein analyzing the tissue region of interest includes processing the raw laser speckle image and the raw hyperspectral image.

30. The method of claim 29, wherein analyzing the tissue region of interest includes overlaying at least two of a plurality of processed data images and/or a plurality of raw data images; and transmitting the overlay to a screen of the mobile device for display.

31. The method of claim 28, wherein the hyperspectral image sensor is a snapshot hyperspectral imager that acquires at least one of the laser speckle image data and a video recording.
